(19) United States
(12) Patent Application Publication
(10) Pub. No.: US 2025/0177147 A1
(43) Pub. Date: Jun. 5, 2025

(54) SYSTEMS, COMPOSITIONS, AND METHODS FOR MAKING AND USING IMPLANTS

(71) Applicant: 3D Orthobiologic Solutions LLC, Herndon, VA (US)

(72) Inventors: Ratna Prakash Kolli, Herndon, VA (US); Benjamin Blair Holmes, Washington, DC (US); Jordan Rashawn Givens, Fort Belvoir, VA (US)

(21) Appl. No.: 19/045,065

(22) Filed: Feb. 4, 2025

Related U.S. Application Data
(63) Continuation of application No. PCT/US23/71602, filed on Aug. 3, 2023.
(60) Provisional application No. 63/370,518, filed on Aug. 5, 2022.

(51) Int. Cl. A61F 2/28; A61F 2/30

(52) U.S. Cl. CPC A61F 2/2875; A61F 2/2803; A61F 2/2846; A61F 2/30771; A61F 2002/285; A61F 2002/2878; A61F 2002/2885; A61F 2002/30062; A61F 2002/30107; A61F 2002/3092

(57) ABSTRACT

In some aspects, the present disclosure provides an implant. In some embodiments, the implant comprises a porous body comprising a form that fills a defect of a bone of a head of a subject. In some embodiments, the implant comprises a recess in the porous body, wherein the recess comprises a dimension larger than an average pore dimension of the porous body. In some embodiments, the implant comprises a biologic disposed in the recess. In some embodiments, the implant comprises an extension.

### CROSS-REFERENCE

This application is a continuation of International Application No. PCT/US23/71602, filed Aug. 3, 2023, which application claims the benefit of U.S. Provisional Application No. 63/370,518, filed Aug. 5, 2022, which is incorporated herein by reference in its entirety.

### BACKGROUND

Implants are sometimes used to reconstruct the morphology of bone and cartilage that result from trauma, tumors, or abnormal bone developments. Engineering an implant for bone reconstruction of crancial and facial bones can present a number of challenges. Cranial and facial bones comprise a variety of shapes and sizes, some of which are irregular in shape, and some of which are non-uniform in thickness. Furthermore, defects in the cranial and facial bones can be heterogenous, because defects can originate from various forms of physical trauma or diseases. Thus, an implant purposed for bone reconstruction in surgery of cranial and facial bones can be challenging to make with a proper size and shape, such that the end-result of the surgery provides an aesthetically pleasing or otherwise aesthetically ordinary appearance for a patient receiving the implant.

Engineering an implant for bone reconstruction of cranial and facial bones can present challenges for ensuring proper fixation of the implant to the native bone surrounding the implant site. During surgical implantation, the placement of fixtures for fixing the implant to the surrounding native bone can present unique challenges for each specific case, as the shapes and sizes of the cranial and facial bones of a subject can vary significantly (e.g., between different areas of the cranium or the face, and between different subjects). To illustrate, some bones in the head are thinner and can be relatively more fragile than others (e.g., the lacrimal bone that forms a part of the eye socket can be as thin as 0.1 mm). Fixtures configured for coupling an implant to be placed at or in proximity to fragile regions can need to be designed with caution, such that complications arising in surgery are minimized and the durability of the coupling between implant and the native bone is sufficient to ensure proper healing after the surgery.

Engineering an implant for bone reconstruction of crancial and facial bones can present challenges for ensuring proper integration of the implant to the native bone surrounding the implant site. An implant that is configured to allow bone regrowth into the space formed by the implant can provide stronger bonds between the implant and the surrounding native bone.

There is a need for improved implants for bone reconstruction and methods of making implants for bone reconstruction.

### SUMMARY

In some aspects, the present disclosure provides an implant, comprising: a porous body comprising a form that fills a defect of a bone of a head of a subject; a recess in the porous body, wherein the recess comprises a dimension larger than an average pore dimension of the porous body; and a biologic disposed in the recess.

In some embodiments, the biologic comprises a biodegradable ceramic.

In some embodiments, the biodegradable ceramic is beta-tricalcium phosphate (beta-TCP).

In some embodiments, the biologic comprises demineralized bone matrix, morselized bone, bone marrow aspirate, bone morphogenetic protein 2 (BMP2), beta-TCP, or any combination thereof.

In some embodiments, two most distant points on a surface of the recess are separated by at least 7 millimeters.

In some embodiments, two nearest points on a surface of the recess are separated by at most 2 millimeters.

In some embodiments, the recess comprises a curved surface.

In some embodiments, the recess forms a channel from one end of the porous body to another end of the porous body.

In some embodiments, the recess comprises an opening that opens inwards to the subject.

In some embodiments, the recess comprises a plurality of recesses.

In some embodiments, each of the plurality of recesses comprises different dimensions.

In some embodiments, the bone is a mandible of the head of the subject.

In some embodiments, the bone is a maxilla of the head of the subject.

In some embodiments, the bone is a nasal bone of the head of the subject.

In some embodiments, the bone is an orbital bone of the head of the subject.

In some embodiments, the bone is a cranial bone of the head of the subject.

In some embodiments, the porous body comprises a chevron hole.

In some embodiments, the porous body comprises a porosity of at least 25, 30, 35, 40, 45, 50, 55, 60, or 65%.

In some aspects, the present disclosure provides an implant, comprising: a porous body comprising a form that fills a defect of a bone of a head of a subject; a biologic disposed in the porous body; and an extension connected to the porous body, wherein the extension is configured to lay against the bone of the head of the subject.

In some embodiments, the bone is a mandible of the head of the subject.

In some embodiments, the extension is configured to lay at least 2 mm above a mandibular division of a trigeminal nerve of the head of the subject.

In some embodiments, the bone is an orbital bone of the head of the subject.

In some embodiments, the extension is configured to lay against a frontal bone of the orbital bone.

In some embodiments, the extension is configured to lay against a maxilla of the orbital bone.

In some embodiments, the extension is configured to lay against a lacrimal bone of the orbital bone.

In some embodiments, the bone is a maxilla of the head of the subject.

In some embodiments, the bone is a nasal bone of the head of the subject.

In some embodiments, the bone is a cranial bone of the head of the subject.

In some embodiments, the extension comprises extends from the porous body by at least 2 millimeters.

In some embodiments, the extension comprises a thickness of at least about 0.8 millimeters.

In some embodiments, the extension is substantially parallel to a surface of the porous body.

In some embodiments, the extension is at an angle to a surface of the porous body.

In some embodiments, the angle is at least 5 degrees.

In some embodiments, the extension comprises an attachment opening.

In some embodiments, the extension comprises a plurality of attachment openings.

In some embodiments, the plurality of attachment openings are oriented at an angle with respect to another.

In some embodiments, the attachment opening is pitched.

In some embodiments, the extension comprises a rounded edge.

In some embodiments, the implant comprises a plurality of extensions.

In some embodiments, the biologic comprises a biodegradable ceramic.

In some embodiments, the biodegradable ceramic is beta-TCP.

In some embodiments, the biologic comprises demineralized bone matrix, morselized bone, bone marrow aspirate, BMP2, or any combination thereof.

In some embodiments, the porous body comprises a porosity of at least 25, 30, 35, 40, 45, 50, 55, 60, or 65%.

In some aspects, the present disclosure provides an implant, comprising: a porous body comprising a form that fills a defect of a bone of a head of a subject, wherein the porous body comprises a biodegradable synthetic polymer; and an extension connected to the porous body, wherein the extension is configured to lay against the bone of the subject.

In some embodiments, the biodegradable synthetic polymer is polycaprolactone.

In some embodiments, the bone is a mandible of the head of the subject.

In some embodiments, the extension is configured to lay at least 2 mm above a mandibular division of a trigeminal nerve of the head of the subject.

In some embodiments, the bone is an orbital bone of the head of the subject.

In some embodiments, the extension is configured to lay against a frontal bone of the orbital bone.

In some embodiments, the extension is configured to lay against a maxilla of the orbital bone.

In some embodiments, the extension is configured to lay against a lacrimal bone of the orbital bone.

In some embodiments, the bone is a maxilla of the head of the subject.

In some embodiments, the bone is a nasal bone of the head of the subject.

In some embodiments, the bone is a cranial bone of the head of the subject.

In some embodiments, the extension comprises extends from the porous body by at least 2 millimeters.

In some embodiments, the extension comprises a thickness of at least about 0.8 millimeters.

In some embodiments, the extension is substantially parallel to a surface of the porous body.

In some embodiments, the extension is at an angle to a surface of the porous body.

In some embodiments, the extension comprises an attachment opening.

In some embodiments, the extension comprises a plurality of attachment openings.

In some embodiments, the attachment opening is pitched.

In some embodiments, the implant comprises a plurality of extensions.

In some embodiments, the biologic comprises a biodegradable ceramic.

In some embodiments, the biodegradable ceramic is beta-TCP.

In some embodiments, the biologic comprises demineralized bone matrix, morselized bone, bone marrow aspirate, BMP2, or any combination thereof.

In some embodiments, the porous body comprises a porosity of at least 25, 30, 35, 40, 45, 50, 55, 60, or 65%.

In some aspects, the present disclosure provides a kit comprising a implant disclosed herein.

In some aspects, the present disclosure provides a method comprising implanting an implant disclosed herein into the head of the subject.

In some embodiments, the implanting comprises coupling the implant to the bone of the subject by installing an attachment device through the implant and the bone of the subject.

In some embodiments, the attachment device is a screw.

In some embodiments, the implant comprises an extension comprising an attachment opening for installing the attachment device.

In some embodiments, the attachment device comprises a plurality of attachment devices.

In some aspects, the present disclosure provides a method of implanting an implant, comprising: providing the implant comprising a porous body that fills a defect of a bone of a head of a subject, wherein the porous body comprises a biologic; and coupling the implant to the bone of the subject by installing an attachment device through the implant and the bone of the subject, wherein the attachment device comprises a non-corrosive material.

In some embodiments, the biologic comprises a biodegradable ceramic.

In some embodiments, the biodegradable ceramic is beta-TCP.

In some embodiments, the biologic comprises demineralized bone matrix, morselized bone, bone marrow aspirate, BMP2, or any combination thereof.

In some embodiments, the non-corrosive material comprises a non-corrosive metal.

In some embodiments, the non-corrosive metal is titanium.

In some embodiments, the bone is a mandible of the head of the subject.

In some embodiments, the bone is a maxilla of the head of the subject.

In some embodiments, the bone is a nasal bone of the head of the subject.

In some embodiments, the bone is an orbital bone of the head of the subject.

In some embodiments, the bone is a cranial bone of the head of the subject.

In some aspects, the present disclosure provides a method of implanting an implant, comprising: providing the implant comprising a porous body that fills a defect of a bone of a head of a subject, wherein the porous body comprises a biodegradable synthetic polymer; and coupling the implant to the bone of the subject by installing an attachment device through the implant and the bone of the subject, wherein the attachment device comprises a non-corrosive material.

In some embodiments, the biodegradable synthetic polymer is polycaprolactone.

In some embodiments, the non-corrosive material comprises a non-corrosive metal.

In some embodiments, the non-corrosive metal is titanium.

In some embodiments, the implant comprises an extension comprising an attachment opening for installing the attachment device.

In some embodiments, the attachment opening is pitched.

In some embodiments, the attachment device is a screw.

In some embodiments, the method further comprises coupling the implant to the bone of the subject by installing a plurality of attachment devices through the implant and the bone of the subject.

In some embodiments, the bone is a mandible of the head of the subject.

In some embodiments, the bone is a maxilla of the head of the subject.

In some embodiments, the bone is a nasal bone of the head of the subject.

In some embodiments, the bone is an orbital bone of the head of the subject.

In some embodiments, the bone is a cranial bone of the head of the subject.

In some aspects, the present disclosure provides a method of implanting an implant, comprising: providing the implant comprising a porous body that fills a defect of a bone of a head of a subject, wherein the porous body comprises a porosity of at least 25%; and coupling the implant to the bone of the subject by installing an attachment device through the implant and the bone of the subject, wherein the attachment device comprises a non-corrosive material.

In some embodiments, the porosity is at least 30, 35, 40, 45, 50, 55, 60, or 65%.

In some embodiments, the non-corrosive material comprises a non-corrosive metal.

In some embodiments, the non-corrosive metal is titanium.

In some embodiments, the implant comprises an extension comprising an attachment opening for installing the attachment device.

In some embodiments, the attachment opening is pitched.

In some embodiments, the attachment device is a screw.

In some embodiments, the attachment device is a plate.

In some embodiments, the method further comprises coupling the implant to the bone of the subject by installing a plurality of attachment devices through the implant and the bone of the subject.

In some embodiments, the bone is a mandible of the head of the subject.

In some embodiments, the bone is a maxilla of the head of the subject.

In some embodiments, the bone is a nasal bone of the head of the subject.

In some embodiments, the bone is an orbital bone of the head of the subject.

In some embodiments, the bone is a cranial bone of the head of the subject.

In some aspects, the present disclosure provides a kit comprising: an implant comprising a porous body that fills a defect of a bone of a head of a subject, wherein the porous body comprises a biologic; and an attachment device comprising a non-corrosive material.

In some embodiments, the biologic comprises a biodegradable ceramic.

In some embodiments, the biodegradable ceramic is beta-TCP.

In some embodiments, the biologic comprises demineralized bone matrix, morselized bone, bone marrow aspirate, BMP2, or any combination thereof.

In some embodiments, the non-corrosive material comprises a non-corrosive metal.

In some embodiments, the non-corrosive metal is titanium.

In some embodiments, the bone is a mandible of the head of the subject.

In some embodiments, the bone is a maxilla of the head of the subject.

In some embodiments, the bone is a nasal bone of the head of the subject.

In some embodiments, the bone is an orbital bone of the head of the subject.

In some embodiments, the bone is a cranial bone of the head of the subject.

In some embodiments, the implant and the attachment device are provided in a hermetically sealed container.

In some embodiments, the kit further comprises an identifier for the subject.

In some embodiments, the identifier comprise a barcode or a quick response (QR) code.

In some aspects, the present disclosure provides a kit comprising: an implant comprising a porous body that fills a defect of a bone of a head of a subject, wherein the porous body comprises a biodegradable synthetic polymer; and an attachment device through the implant and the bone of the subject, wherein the attachment device comprises a non-corrosive material.

In some embodiments, the biodegradable synthetic polymer is polycaprolactone.

In some embodiments, the non-corrosive material comprises a non-corrosive metal.

In some embodiments, the non-corrosive metal is titanium.

In some embodiments, the implant comprises an extension comprising an attachment opening for installing the attachment device.

In some embodiments, the attachment opening is pitched.

In some embodiments, the attachment device is a screw.

In some embodiments, the kit further comprises a plurality of attachment devices.

In some embodiments, the bone is a mandible of the head of the subject.

In some embodiments, the bone is a maxilla of the head of the subject.

In some embodiments, the bone is a nasal bone of the head of the subject.

In some embodiments, the bone is an orbital bone of the head of the subject.

In some embodiments, the bone is a cranial bone of the head of the subject.

In some embodiments, the implant and the attachment device are provided in a hermetically sealed container.

In some embodiments, the kit further comprises an identifier for the subject.

In some embodiments, the identifier comprise a barcode or a quick response (QR) code.

In some aspects, the present disclosure provides a kit comprising: an implant comprising a porous body that fills a defect of a bone of a head of a subject, wherein the porous body comprises a porosity of at least 25%; and an attachment device through the implant and the bone of the subject, wherein the attachment device comprises a non-corrosive material.

In some embodiments, the porosity is at least 30, 35, 40, 45, 50, 55, 60, or 65%.

In some embodiments, the non-corrosive material comprises a non-corrosive metal.

In some embodiments, the non-corrosive metal is titanium.

In some embodiments, the implant comprises an extension comprising an attachment opening for installing the attachment device.

In some embodiments, the attachment opening is pitched.

In some embodiments, the attachment device is a screw.

In some embodiments, the attachment device is a plate.

In some embodiments, the kit further comprises a plurality of attachment devices.

In some embodiments, the bone is a mandible of the head of the subject.

In some embodiments, the bone is a maxilla of the head of the subject.

In some embodiments, the bone is a nasal bone of the head of the subject.

In some embodiments, the bone is an orbital bone of the head of the subject.

In some embodiments, the bone is a cranial bone of the head of the subject.

In some embodiments, the implant and the attachment device are provided in a hermetically sealed container.

In some embodiments, the kit further comprises an identifier for the subject.

In some embodiments, the identifier comprise a barcode or a quick response (QR) code.

In some aspects, the present disclosure provides a method of making an implant, comprising: obtaining an electronic three-dimensional representation of a bone of a subject; removing noise from the electronic three-dimensional representation of the bone of the subject to generate a denoised electronic three-dimensional representation; creating an implant electronic three-dimensional representation that complements the denoised electronic three-dimensional representation; and using the implant electronic three-dimensional representation to generate the implant.

In some embodiments, the method further comprises, after (c), electronically designing an attachment opening, an extension, or a recess in the implant electronic three-dimensional representation.

In some embodiments, a position of the attachment opening, the extension, or the recess is selected based on: a thickness of the bone, a fragility of the bone, an absence of a nerve in the subject proximal to the position, an expected torque at a location proximal to the position during installation of an attachment at the position, or any combination thereof.

In some embodiments, the implant is generated using 3-dimensional (3-D) printing.

In some embodiments, the 3-D printing comprises layer-by-layer printing.

In some embodiments, a longitudinal axis of the attachment opening is substantially parallel to a normal of a plane of a layer in the implant printed by the layer-by-layer printing.

In some embodiments, an angle between the longitudinal axis and the normal is less than about 45, 30, 15, 10, 5, 4, 3, 2, or 1 degrees.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1F show implants for a mandible.

FIGS. 2A-2B show implants for a mandible.

FIGS. 3A-3D show implants for an orbital bone.

FIGS. 4A-4E show implants for a maxilla and nasal bone.

FIGS. 5A-5C show implants for a cranial bone.

FIGS. 6A-6D provide an overview for making a implant provided herein. FIG. 6A shows a method for generating an implant. FIGS. 6B-6D show 3-dimensional (3D) electronic representations of a bone and an implant.

FIG. 7 shows a computer system.

FIG. 8 shows a kit.

FIGS. 9A-9B show bones.

### DETAILED DESCRIPTION

In general, systems and methods are disclosed herein for making and using implants for bone reconstruction in surgery of cranial and facial bones. Provided herein is a platform for making and using implants that are case-specific to the needs of individual subjects, e.g., patients, and preferences of individual healthcare providers, e.g., surgeons, dentists, otorhinolaryngologists, otolaryngologists, head and neck surgeons, ENT surgeons, orthodontists, neurosurgeons, ophthalmologists, orthopaedic surgeons, pediatric surgeons, plastic surgeons, maxillofacial surgeons, cosmetic surgeons, or any other medical professional that operates on cranial bones and/or facial bones. In some cases, the systems and methods disclosed herein can provide improved outcomes in terms of physical healing as well as aesthetic recovery.

Provided herein are materials and structures, e.g., biocompatiable materials and structures, and systems and methods for making implants. In some embodiments, the materials and structures, e.g., biocompatible materials and structures comprise a composite material that is 3-D printable. In some cases, the materials and structures, e.g., biocompatible materials and structures permit the printing of case-specific bone reconstruction scaffolds to form a precise size and shape that provides better outcomes (e.g., physical recovery as well as aesthetics) for subjects, e.g., patients, than other scaffolds. In some cases, the implant comprises one or more biologics, e.g., to promote bone regrowth. In some cases, the implant is configured to allow osseointegration and osteoinduction, which results in gradual degradation of the material comprising the implant. In some cases, the implant comprises one or more attachment devices for coupling the implant to a native bone of a subject.

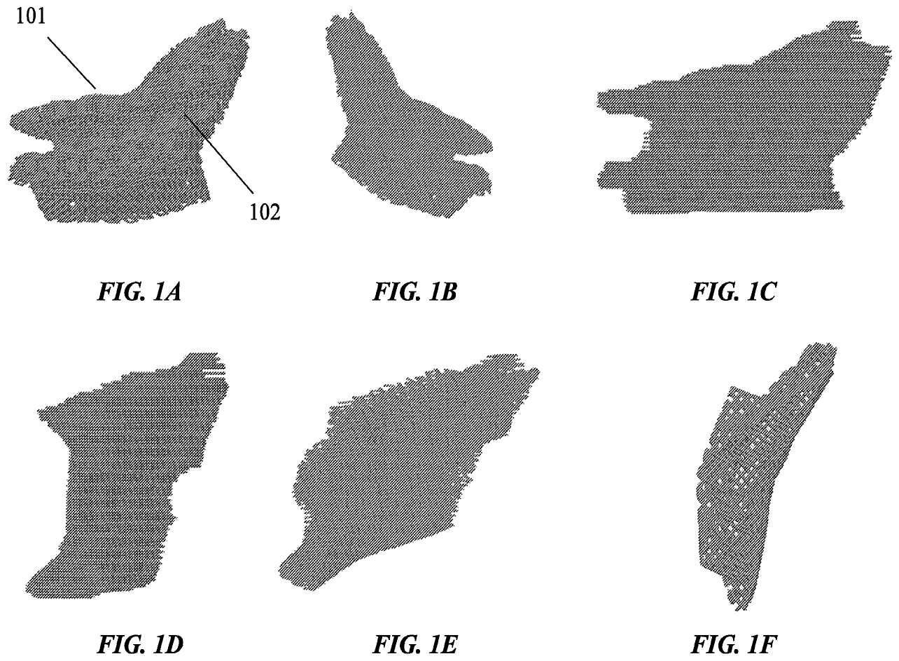

FIGS. 1A-1F provides an overview of one aspect of the implant provided herein. In some aspects, the present disclosure provides an implant (FIG. 1A, 101), comprising: (a) a porous body comprising a form that fills a defect of a bone of a head of a subject; (b) a recess (FIG. 1A, 102) in the porous body, wherein the recess comprises a dimension larger than an average pore dimension of the porous body; and (c) a biologic disposed in the recess. The porous body can be configured to provide space for cells from the subject to grow into, which can improve osseointegration and osteoinduction of the implant into the subject's body.

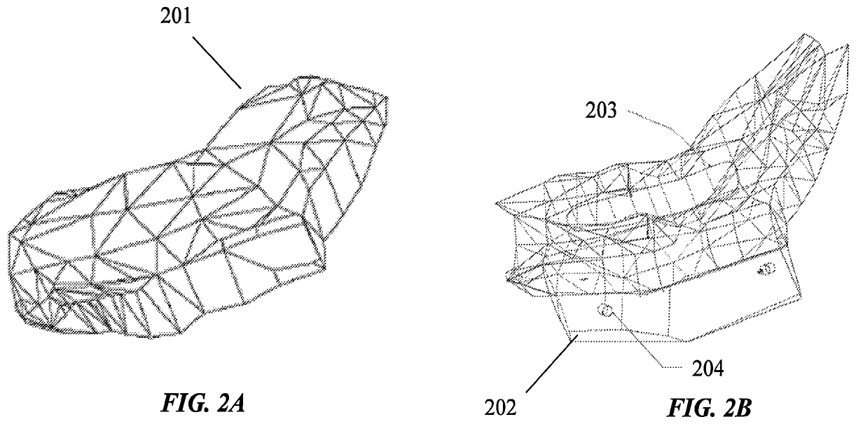

FIGS. 2A-2B provides an overview of an additional aspect of the implant provided herein. In some aspects, the present disclosure provides an implant, comprising: (a) a porous body (201) comprising a form that fills a defect of a bone of a head of a subject; (b) a biologic disposed in the porous body; and (c) an extension (202) connected to the porous body, wherein the extension is configured to lay against the bone of the head of the subject. The extension can be configured to couple to the bone of the head of the subject via an attachment device. The position of the extension can be based on case-specific details for the subject or healthcare provider, e.g., surgeon, preferences. In some embodiments, porous body comprises a recess (203). The recess can have a dimension of about 3.5 millimeters (mm). The recess can have a variety of dimensions. In some embodiments, the recess has a dimension of at least 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 15, 20, 25, 30, 35, 40, 45, or 50 mm. In some embodiments, the recess has a dimension of at most 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 15, 20, 25, 30, 35, 40, 45, or 50 mm. In some embodiments, the recess has a dimension from 0.1 to 0.5, from 0.2 to 0.6, from 0.3 to 0.7, from 0.4 to 0.8, from 0.5 to 0.9, from 0.6 to 1, from 0.7 to 1.5, from 0.8 to 2, from 0.9 to 2.5, from 1 to 3, from 1.5 to 3.5, from 2 to 4, from 2.5 to 4.5, from 3 to 5, from 3.5 to 6, from 4 to 7, from 4.5 to 8, from 5 to 9, from 6 to 10, from 7 to 15, from 8 to 20, from 9 to 25, from 10 to 30, from 15 to 35, from 20 to 40, from 25 to 45, or from 30 to 50 mm. In some embodiments, the shape and/or the dimensions of the recess can vary based on the shape and/or dimensions of the implant. For example, the dimension of the recess can vary when the topography of the implant is inconsistent. In some embodiments, the extension can have a hole (204) for coupling the implant to the bone of the head of the subject. The hole can have a diameter of about 0.8 mm. The hole can have a variety of dimensions. In some embodiments, the hole has a diameter of at least 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9, or 10 mm. In some embodiments, the hole has a diameter of at most 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9, or 10 mm. In some embodiments, the hole has a diameter from 0.1 to 0.5, from 0.2 to 0.6, from 0.3 to 0.7, from 0.4 to 0.8, from 0.5 to 0.9, from 0.6 to 1, from 0.7 to 1.5, from 0.8 to 2, from 0.9 to 2.5, from 1 to 3, from 1.5 to 3.5, from 2 to 4, from 2.5 to 4.5, from 3 to 5, from 3.5 to 6, from 4 to 7, from 4.5 to 8, from 5 to 9, or from 6 to 10 mm.

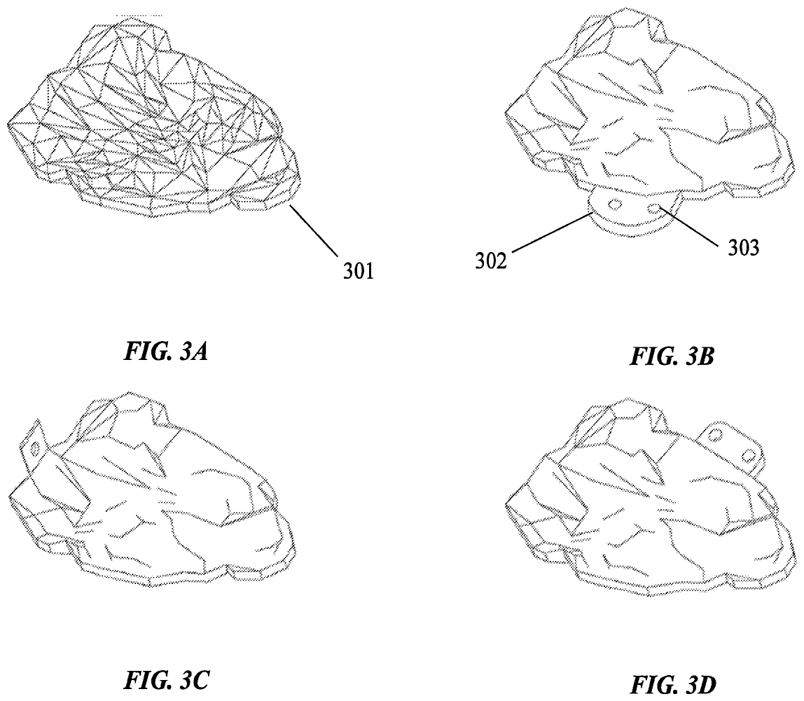

FIGS. 3A-3D provides an overview of an additional aspect of the implant provided herein. In some aspects, the present disclosure provides an implant, comprising: (a) a porous body (301) comprising a form that fills a defect of a bone of a head of a subject, wherein the porous body comprises a biodegradable synthetic polymer; and (b) an extension (302) connected to the porous body, wherein the extension is configured to lay against the bone of the subject. The biodegradable synthetic polymer can improve integration of the implant by the cells of the subject. The extension can comprise various numbers of holes. The extension can comprise 1, 2, 3, 4, 5, 6, 7, 8, 9, 10, or more holes (303). One or more attachment devices can attach the implant to the bone of the head of the subject through the one or more holes. For example, two or more fixation screws can be attach the implant to the bone of the head of the subject, creating stability to the implant, as shown in FIG. 3B. The extension and the one or more holes can be positioned to minimize or prevent disturbance in the natural movement or appearance of the head of the subject. In some embodiments, the extension is configured for placement against the nasal bone of the head of the subject, for example, as shown in FIG. 3C. In some embodiments, the extension is configured for placement towards the back of the eye, as shown in FIG. 3D.

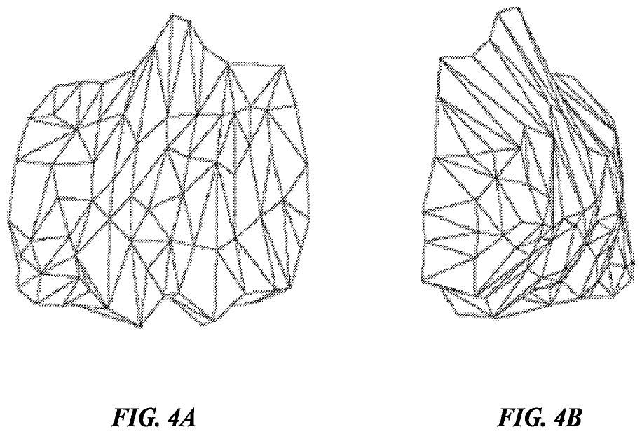

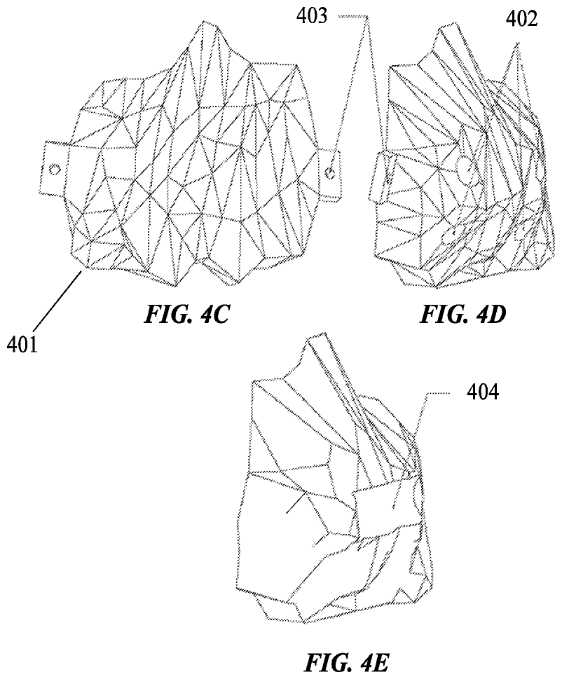

FIGS. 4A-4E provides an overview of an additional aspect of the implant provided herein. In some aspects, the present disclosure provides a method of implanting an implant, comprising: (a) providing the implant comprising a porous body (FIG. 4C, 401) that fills a defect of a bone of a head of a subject, wherein the porous body comprises a biologic; and (b) coupling the implant to the bone of the subject by installing an attachment device through the implant and the bone of the subject, wherein the attachment device comprises a non-corrosive material. In some embodiments, the attachment device is a crew that is installed through a screw hole (FIGS. 4C and 4D, 403) of an extension. The extension can comprise various number of screw holes. The extension can comprise 1, 2, 3, 4, 5, 6, 7, 8, 9, 10, or more screw holes. The implant can be coupled to the bone of the subject using 1, 2, 3, 4, 5, 6, 7, 8, 9, 10, or more attachment devices. In some embodiments, the implant comprises one or more recesses (FIG. 4D, 402) that comprises biologic. The implant can comprise 1, 2, 3, 4, 5, 6, 7, 8, 9, 10, or more recesses. In some embodiments, a recess comprises an exposed opening (FIG. 4E, 404) facing inwards towards the center of the subject's head, for example, as shown in FIG. 4E. In some embodiments, a recess comprises an exposed opening facing outwards from the center of the subject's head. Non-corrosive metals can be used to provide chemically safe and sturdy attachment devices between the implant and the subject's bone.

In some aspects, the present disclosure provides a method of implanting an implant, comprising: (a) providing the implant comprising a porous body that fills a defect of a bone of a head of a subject, wherein the porous body comprises a biodegradable synthetic polymer; and (b) coupling the implant to the bone of the subject by installing an attachment device through the implant and the bone of the subject, wherein the attachment device comprises a non-corrosive material.

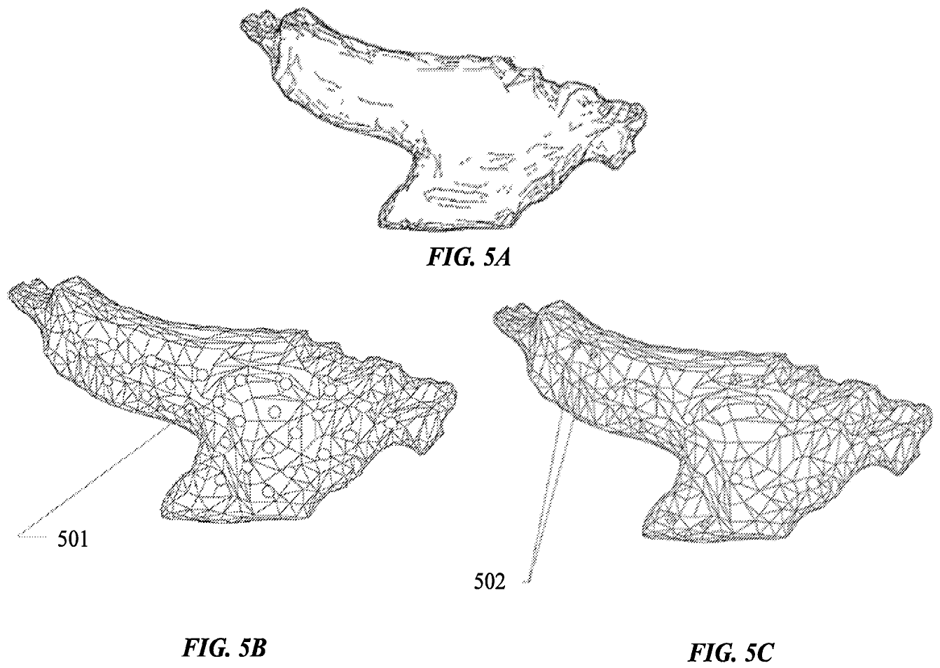

FIGS. 5A-5C provides an overview of an additional aspect of the implant provided herein. In some aspects, the present disclosure provides an implant, comprising: (a) a porous body comprising a form that fills a defect of a bone of a head of a subject, wherein the porous body comprises a pressure relief hole (FIG. 5B, 501); and (b) a hole (FIG. 5C, 502) for attaching the implant to the bone of the head of the subject using an attachment device. The pressure relief hole can reduce or prevent build-up of pressure when the implant is implanted in the head of the subject. The pressure relief hole can comprise a chevron shape. The chevron shape can be oriented such that a pointed end of the chevron hole is oriented towards the center of the subject's head. The chevron shape can be oriented such that a pointed end of the chevron hole is oriented away from the center of the subject's head. The porous body can have various numbers of pressure relief holes. The porous body can comprise at least 1, 2, 3, 4, 5, 6, 7, 8, 9, 10, 15, 20, 25, 30, 35, 40, 45, 50 pressure relief holes. The porous body can comprise at most 1, 2, 3, 4, 5, 6, 7, 8, 9, 10, 15, 20, 25, 30, 35, 40, 45, 50 pressure relief holes. For example, the porous body can comprise from 20 to 30 pressure relief holes, as shown in FIG. 5B. In another example, the porous body can comprise from 5 to 10 pressure relief holes, as shown in FIG. 5C. The position of the pressure relief hole can be based on case-specific details of the subject and healthcare provider, e.g., surgeon's, preferences. The porous body can comprise various numbers of holes. In some embodiments, the porous body comprises at least 1, 2, 3, 4, 5, 6, 7, 8, 9, or 10 holes for attaching the implant to the bone of the head of the subject using at least 1, 2, 3, 4, 5, 6, 7, 8, 9, or 10 attachment devices. In some embodiments, the porous body comprises at most 1, 2, 3, 4, 5, 6, 7, 8, 9, or 10 holes for attaching the implant to the bone of the head of the subject using at most 1, 2, 3, 4, 5, 6, 7, 8, 9, or 10 attachment devices. In some embodiments, a hole can comprise a non-circular cross-sectional profile.

FIGS. 1A-1F provides an overview of an additional aspect of the implant provided herein. In some aspects, the present disclosure provides a method of implanting an implant, comprising: (a) providing the implant comprising a porous body that fills a defect of a bone of a head of a subject, wherein the porous body comprises a porosity of at least 25%; and (b) coupling the implant to the bone of the subject by installing an attachment device through the implant and the bone of the subject, wherein the attachment device comprises a non-corrosive material. The porosity of the porous body can be based on case-specific details of the subject and healthcare provider, e.g., surgeon's, preferences. For example, implants that are expected to experience high stress after implantation can comprise higher porosities than implants that are expected to experience lower stress.

### Methods of Making

In some aspects, the present disclosure provides a method of making an implant. In some embodiments, the method comprises obtaining an electronic three-dimensional representation of a bone of a subject. The electronic three-dimensional representation can be obtained using any of a variety of instruments. In some embodiments, the electronic three-dimensional representation is an X-Ray representation, a magnetic resonance imaging (MRI) representation, a positron emission tomography (PET) representation, or a computed tomography (CT) representation. The electronic three-dimensional representation can be provided in any of a variety of file formats including Analyze format, Nifti format, Mine format, a dcm format, or DICOM format.

In some embodiments, the method comprises removing noise from the electronic three-dimensional representation of the bone of the subject to generate a denoised electronic three-dimensional representation. Removing the noise can comprise removing high frequency features from the electronic three-dimensional representation of the bone. Removing the noise can comprise removing a sharp edge from the electronic three-dimensional representation of the bone. In some embodiments, the method comprises removing a representation of a non-defective bone from the electronic three-dimensional representation of the bone. In some embodiments, the method comprises removing a representation of a non-defective portion of bone from the electronic three-dimensional representation of the bone. In some cases, the method does not comprise removing noise from the electronic three-dimensional representation of the bone.

In some embodiments, the method comprises creating an implant electronic three-dimensional representation that complements the denoised electronic three-dimensional representation of the bone. The implant electronic three-dimensional representation can be configured to fill a representation of a defect in the denoised electronic three-dimensional representation. The implant electronic three-dimensional representation can complement a surface of a representation of a defective portion of a bone in the denoised electronic three-dimensional representation. The implant electronic three-dimensional representation can be configured to complete a missing portion of bone in a representation of a defect in the denoised electronic three-dimensional representation, e.g., like a puzzle piece.

In some embodiments, the method comprises using the implant electronic three-dimensional representation to generate the implant. In some embodiments, the method comprises using the implant electronic three-dimensional representation to 3D print the implant. The 3D printing can be constructive or reductive printing. In some embodiments, the method comprises using the implant electronic three-dimensional representation to make a mold of the implant. In some embodiments, the method comprises using the implant electronic three-dimensional representation to make an die of the implant. In some embodiments, the mold or the die is used to make the implant. The 3D printing can be layer by layer printing. A longitudinal axis of the attachment opening can be substantially parallel to a normal of a plane of a layer in the implant printed by the layer-by-layer printing. An angle between the longitudinal axis and the normal can be less than about 45, 30, 15, 10, 5, 4, 3, 2, or 1 degrees.

In some embodiments, the method comprises creating a mold or die based on the denoised electronic three-dimensional representation of the bone, or an electronic three-dimensional representation of the bone that has not been denoised. The mold or die can be used to make the implant. The implant can be 3D printed on the mold or die. The implant can be made to fill a representation of a defect in the mold or die. The implant can be made to complement a surface of a representation of a defective portion of a bone mold or die. The implant can be made to complete a missing portion of bone in a representation of a defect in the mold or die.

In some embodiments, the method comprises electronically designing an attachment opening, an extension, or a recess in the implant electronic three-dimensional representation. In some embodiments, the method comprises electronically designing a plurality of attachment openings, a plurality of extensions, or a plurality of recesses in the implant electronic three-dimensional representation. The size, form, and the position of the attachment opening, the extension, or the recess can be designed based on case-specific details for the subject or a healthcare provider's, e.g., surgeon's, preferences. A position of the attachment opening, the extension, or the recess can be selected based on: a thickness of the bone, a fragility of the bone, an absence of a nerve in the subject proximal to the position, an expected torque at a location proximal to the position during installation of an attachment at the position, or any combination thereof. In some embodiments, the method comprises designing and or including an attachment, opening, extension, or a recess in an implant without, e.g., without electronically designing the attachment, opening, extension, or recess.

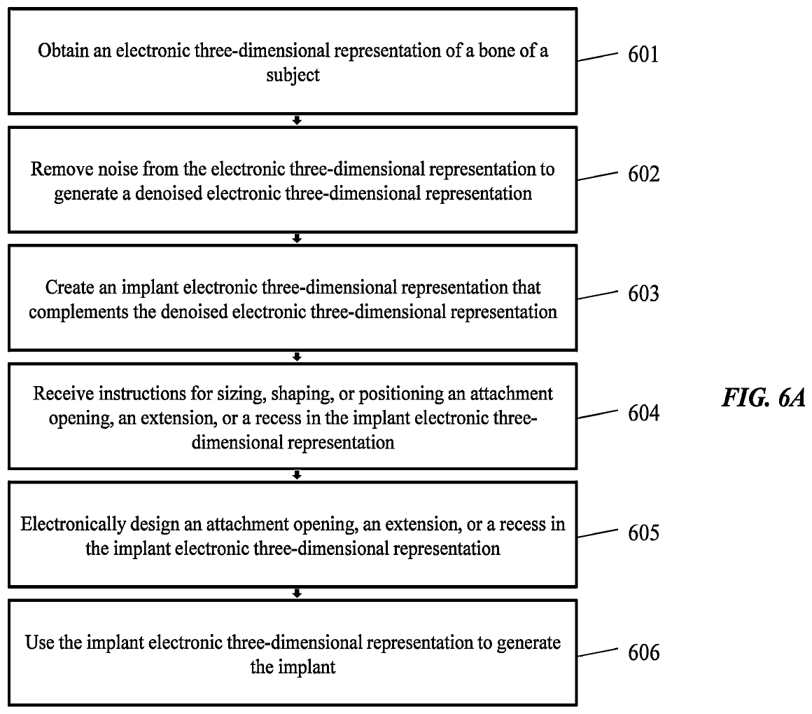

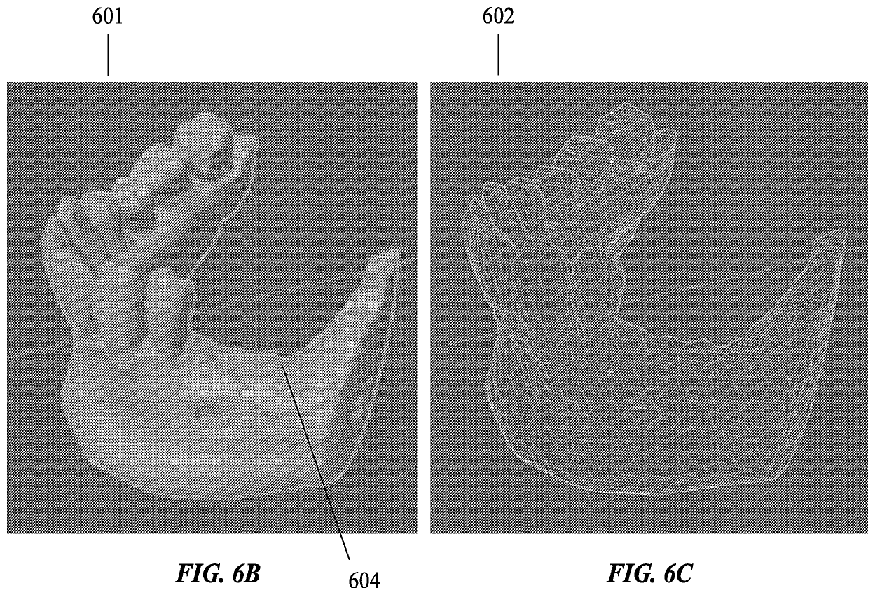

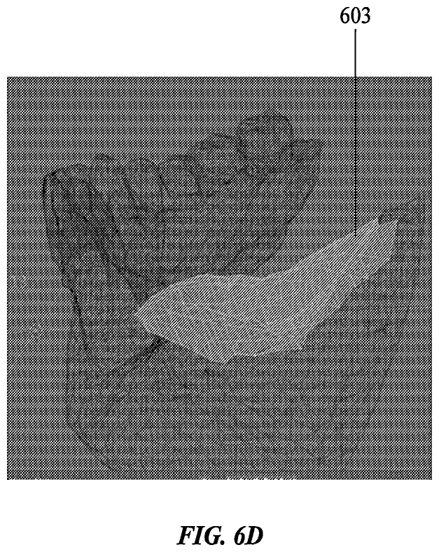

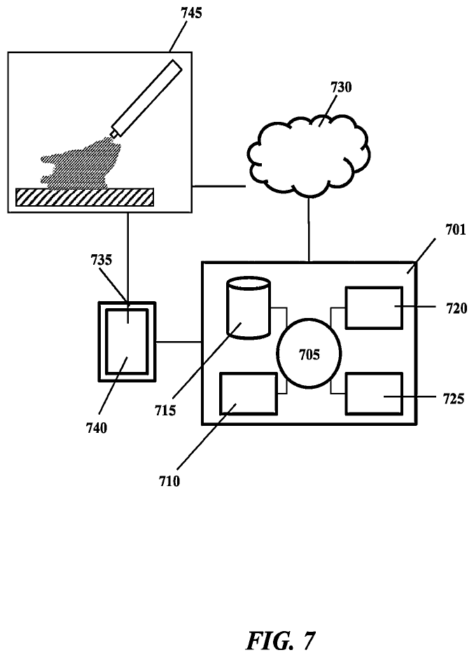

In some aspects, the present disclosure provides a method of making an implant. FIGS. 6A-6D provide an overview for making a implant provided herein. In some embodiments, the method comprises obtaining an electronic three-dimensional representation (601) of a bone of a subject. In some embodiments, the method comprises removing noise from the electronic three-dimensional representation of the bone of the subject to generate a denoised electronic three-dimensional representation (602). In some embodiments, the method comprises creating a implant electronic three-dimensional representation (603) that complements the denoised electronic three-dimensional representation. In some embodiments, the method comprises receiving instructions for sizing, shaping, or positioning an attachment opening, an extension, or a recess in the implant electronic three-dimensional representation (604). In some embodiments, the method comprises electronically designing an attachment opening, an extension, or a recess in the implant electronic three-dimensional representation (605). In some embodiments, the method comprises fabricating the implant based on the implant electronic three-dimensional representation (606). In some embodiments, as shown in FIG. 7, the fabricating comprises using a 3D printer (745) to print the implant.

In some embodiments, a position of the attachment opening, the extension, or the recess is selected based on: a thickness of the bone, a fragility of the bone, an absence of a nerve in the subject proximal to the position, an expected torque at a location proximal to the position during installation of an attachment device at the position, or any combination thereof. The position of the attachment opening, the extension, or the recess can be uniquely placed differently between subjects. Various factors can be considered when choosing the position. The morphology and the strength of cranial and facial bones adjacent or in proximity to a defect can vary between subjects, and between areas of the cranium and face. The anatomy of the injury can vary between individual cases, for example, a bullet wound can have different characteristics from a blunt-force trauma, and a surgical wound arising from cancer in one patient can differ from a wound in another subject. The position of the attachment opening, the extension, or the recess can be determined based on healthcare provider, e.g., surgeon, preferences, for example, to improve surgical outcomes when implanting the implant.

### Implants

In some aspects, the present disclosure provides an implant. In some embodiments, the implant comprises a body, e.g., a porous body. In some embodiments, the implant does not comprise a porous body.

In some embodiments, the implant comprises one or more biologics. The one or more biologics can be any biologic described herein. In some embodiments, the biologic is disposed in one or more recesses of the implant. In some embodiments, the implant comprises the biologic, and the biologic is not disposed in a recess of the implant. In some embodiments, the biologic comprises a biodegradable ceramic. In some embodiments, the biodegradable ceramic is beta-tricalcium phosphate (beta-TCP). In some embodiments, the biologic comprises demineralized bone matrix, morselized bone, bone marrow aspirate, bone morphogenetic protein 2 (BMP2), beta-TCP, or any combination thereof.

In some embodiments, the implant comprises one or more recesses in the body, e.g., porous body. The one or more recesses can have any of a variety of dimensions. In some embodiments, two most distant points on a surface of the one or more recesses are separated by at least 7 millimeters. In some embodiments, two nearest points on a surface of the one or more recesses are separated by at most 2 millimeters. In some embodiments, the one or more recesses comprise a dimension larger than an average pore dimension of the porous body. In some embodiments, the average pore dimension is based on a mean, median, or a mode of pores in the porous body. In some embodiments, a pore dimension is based on a distance between two separated points in the porous body. In some embodiments, the distance between two separated points in the porous body is the nearest distance between the two separated points in the porous body. In some embodiments, a pore dimension is based on a size of a hypothetical sphere that can occupy a pore without overlapping with an occupied volume of a material comprising the porous body.

The one or more recesses can have one or more shapes and one or more sizes. For example, in some embodiments, the one or more recesses comprise a curved surface. The curved surface can comprise a profile that forms an arc of a circle. In some embodiments, the one or more recesses form a channel from one end of the porous body to another end of the porous body. In some embodiments, the one or more recesses comprise an opening that opens inwards to the subject when the implant is implanted in a subject. In some cases, the one or more recesses comprise an opening that opens outwards from the subject when the implant is implanted in the subject.

In some embodiments, the one or more recesses comprise a plurality of recesses. In some embodiments, each of the plurality of recesses comprises different dimensions from one another. In some embodiments, each of the plurality of recesses comprise the same dimensions with one another. The implant can have about, at least, or at most 1, 2, 3, 4, 5, 6, 7, 8, 9, or 10 recesses.

The implant can comprise various porosities. Porosity can be defined by the percentage of the volume that is empty space. The porosity of the implant can be based on how much of the volume is not made of the printed material. The porosity can be determined based on the interior fill percentage (infill) and the chosen infill pattern. Porosity can refer to a ratio between a volume of one or more voids to the total volume. Porosity can refer to a ratio between the volume of the one or more voids in a porous body of an implant to the total volume of the implant. The one or more voids can be bounded within a continuous surface having a minimal possible surface area that can surround the porous body. The total volume can be bounded within a continuous surface having a minimal possible surface area that can surround the porous body. Porosity can refer to a ratio between the fluid-occupiable volume (e.g., water) in a porous body of an implant to the total volume of the implant. The fluid-occupiable volume can be measured as the volume of a fluid (e.g., water) that is displaced when porous body is placed in the fluid. In some embodiments, the porous body comprises a porosity of at least 5, 10, 15, 20, 25, 30, 35, 40, 45, 50, 55, 60, 65, 70, 75, 80, 85, 90, or 95%. In some embodiments, the porous body comprises a porosity of at most 5, 10, 15, 20, 25, 30, 35, 40, 45, 50, 55, 60, 65, 70, 75, 80, 85, 90, or 95%. In some embodiments, the porous body comprises a porosity of about 5, 10, 15, 20, 25, 30, 35, 40, 45, 50, 55, 60, 65, 70, 75, 80, 85, 90, or 95%. In some embodiments, the porous body comprises a porosity of from 1 to 10%, from 1 to 20, from 1 to 30, from 1 to 40, from 1 to 50, from 1 to 60, from 1 to 70, from 1 to 80, from 1 to 90, from 1 to 99, from 10 to 20, from 10 to 30, from 10 to 40, from 10 to 50, from 10 to 60, from 10 to 70, from 10 to 80, from 10 to 90, from 10 to 99, from 20 to 30, from 20 to 40, from 20 to 50, from 20 to 60, from 20 to 70, from 20 to 80, from 20 to 90, from 20 to 99, from 30 to 40, from 30 to 50, from 30 to 60, from 30 to 70, from 30 to 80, from 30 to 90, from 30 to 99, from 40 to 50, from 40 to 60, from 40 to 70, from 40 to 80, from 40 to 90, from 40 to 99, from 50 to 60, from 50 to 70, from 50 to 80, from 50 to 90, from 50 to 99, from 60 to 70, from 60 to 80, from 60 to 90, from 60 to 99, from 70 to 80, from 70 to 90, from 70 to 99, from 80 to 90, from 80 to 99, or from 90 to 99%. In some embodiments, the entire implant is porous. In some embodiments, part of the implant is porous. In some embodiments, more than 50% of the implant is porous. In some embodiments, less than 50% of the implant is porous. In some embodiments, implant has a porosity of 25% to 65%.

In some embodiments, the implant comprises one or more holes. The one or more holes can comprise various shapes. The one or more holes can be a chevron hole. The one or more holes can have the shape of a cylinder, a hemisphere, a cone.

In some embodiments, the implant comprises a form that fills a defect of a bone of a subject. In some embodiments, the bone is in a head of a subject. In some embodiments, the bone is a mandible of the head of the subject. In some embodiments, the bone is a maxilla of the head of the subject. In some embodiments, the bone is a nasal bone of the head of the subject. In some embodiments, the bone is an orbital bone of the head of the subject. In some embodiments, the bone is a cranial bone of the head of the subject.

In some embodiments, the implant comprises one or more extensions connected to the body, e.g., porous body. In some embodiments, the one or more extensions are configured to lay against one or more bones of the head of the subject. In some embodiments, the one or more extensions are configured to lay at least 2 mm above a mandibular division of a trigeminal nerve of the head of the subject. In some embodiments, the one or more extensions lay at least 0.5, 1, 1.5, 2, 2.5, 3, 3.5, 4, 4.5, or 5 mm above a mandibular division of a trigeminal nerve of the head of the subject. In some embodiments, the one or more extensions lay at most 0.5, 1, 1.5, 2, 2.5, 3, 3.5, 4, 4.5, or 5 mm above a mandibular division of a trigeminal nerve of the head of the subject. In some embodiments, the one or more extensions are configured to lay flat against the one or more bones. In some embodiments, only a portion of the one or more extensions are configured to lay against the one or more bones.

In some embodiments, the one or more bones is an orbital bone of the head of the subject. In some embodiments, the one or more extensions are configured to lay against a frontal bone of the orbital bone. In some embodiments, the one or more extensions are configured to lay against a maxilla of the orbital bone. In some embodiments, the one or more extensions are configured to lay against a lacrimal bone of the orbital bone. In some embodiments, the one or more extensions extend from the body, e.g., porous body by at least 2 mm. In some embodiments, the one or more extensions extend from the body, e.g., porous body, by at least 0.5, 1, 1.5, 2, 2.5, 3, 3.5, 4, 4.5, or 5 mm. In some embodiments, the one or more extensions extend from the body, e.g., porous body. by at most 0.5, 1, 1.5, 2, 2.5, 3, 3.5, 4, 4.5, or 5 mm. In some embodiments, the extension comprises a thickness of at least about 0.8 millimeters. In some embodiments, the extension comprises a thickness of at least 0.1, 0.2, 0.3, 0.4, 0.5, 0.6, 0.7, 0.8, 0.9, 1, 1.2, 1.4, 1.6, 1.8, or 2 millimeters. In some embodiments, the extension comprises a thickness of at most 0.1, 0.2, 0.3, 0.4, 0.5, 0.6, 0.7, 0.8, 0.9, 1, 1.2, 1.4, 1.6, 1.8, or 2 millimeters. In some embodiments, the extension is substantially parallel to a surface of the porous body. In some embodiments, the extension is at an angle to a surface of the porous body. In some embodiments, the angle is at least 5 degrees. In some embodiments, the angle is at least 1, 2, 3, 4, 5, 10, 15, 30, 45, 60, 75, or 90 degrees. In some embodiments, the angle is at most 1, 2, 3, 4, 5, 10, 15, 30, 45, 60, 75, or 90 degrees. In some embodiments, the extension comprises an attachment opening. In some embodiments, the extension comprises a plurality of attachment openings. In some embodiments, the plurality of attachment openings are oriented at an angle with respect to one another. In some embodiments, the angle is at least 1, 2, 3, 4, 5, 10, 15, 30, 45, 60, 75, or 90 degrees. In some embodiments, the angle is at most 1, 2, 3, 4, 5, 10, 15, 30, 45, 60, 75, or 90 degrees. In some embodiments, the attachment opening is pitched. The attachment opening can have a pitch of at least 0.5, 1, 1.5, 2, 2.5, 3, 3.5, 4, 4.5, or 5 mm. The attachment opening can have a pitch of at most 0.5, 1, 1.5, 2, 2.5, 3, 3.5, 4, 4.5, or 5 mm. In some embodiments, the extension comprises a rounded edge. In some embodiments, the implant comprises a plurality of extensions. The implant can have 1, 2, 3, 4, 5, 6, 7, 8, 9, or 10 or more extensions.

In some embodiments, the implant comprises a biodegradable synthetic polymer. In some embodiments, a portion of the implant comprises a biodegradable synthetic polymer. In some embodiments, the porous body comprises a biodegradable synthetic polymer. In some embodiments, the biodegradable synthetic polymer is a thermosetting plastic. In some embodiments, the biodegradable synthetic polymer is a thermoplastic. A thermoplastic can refer to a plastic material that can be molded, deformed, or become soft when heated above a glass transition temperature of the plastic material. In some embodiments, the implant comprises a melt or a glass of a plurality of individual polymer molecules. A thermosetting plastic can refer to a plastic material that comprises a networked polymer morphology at a molecular level. A thermosetting plastic can be cross-linked to form the networked polymer morphology. A thermosetting plastic can retain its shape when above or below the glass transition temperature. A thermosetting plastic can be deformed and returned to the shape before being deformed, e.g., in elastomers. In some embodiments, the biodegradable synthetic polymer is polycaprolactone. In some embodiments, the implant comprises poly(alpha-hydroxy acids), poly(lactide-co-glycolide) (PLGA), polylactide (PLA), polyglycolide (PG), polyethylene glycol (PEG), conjugates of poly(alpha-hydroxy acids), poly(orthoester)s (POE), polycaprolactone, polyaspirins, polyphosphagenes, collagen, starch, pre-gelatinized starch, hyaluronic acid, chitosans, gelatin, alginates, albumin, fibrin, vitamin E compounds, such as alpha tocopheryl acetate, d-alpha tocopheryl succinate, D,L-lactide, or L-lactide, caprolactone, dextrans, vinylpyrrolidone, polyvinyl alcohol (PVA), PVA-g-PLGA, PEGT-PBT copolymer (polyactive), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate), or combinations thereof.

In some embodiments, the biodegradable synthetic polymer comprises poly(alpha-hydroxy acids), poly(lactide-co-glycolide) (PLGA), polylactide (PLA), polyglycolide (PG), polyethylene glycol (PEG), conjugates of poly(alpha-hydroxy acids), poly(orthoester)s (POE), polycaprolactone, polyaspirins, polyphosphagenes, collagen, starch, pre-gelatinized starch, hyaluronic acid, chitosans, gelatin, alginates, albumin, fibrin, vitamin E compounds, such as alpha tocopheryl acetate, d-alpha tocopheryl succinate, D,L-lactide, or L-lactide, caprolactone, dextrans, vinylpyrrolidone, polyvinyl alcohol (PVA), PVA-g-PLGA, PEGT-PBT copolymer (polyactive), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate), or combinations thereof.

In some embodiments, the implant degrades in the body of the subject when implanted. The implant can degrade over at least 1, 2, 3, 4, 5, 6, or 7 days. The implant can degrade over at least 1, 2, 3, or 4 weeks. The implant can degrade over at least 1, 2, 3, 4, 5, 6, 7, 8, 9, 10, 11, or 12 months. The implant can degrade over at least 1, 2, 3, 4, 5, 6, 7, 8, 9, or 10 years. In some embodiments, the implant partially degrades. In some embodiments, the implant completely degrades.

### Bones

A defect in a bone of a subject can be caused by any of a number of different factors including, but not limited to, trauma, pathological disease, or surgical intervention. A defect in the cranial and facial bones of a subject can compromise stability, protection, and the appearance of a subject. Provided herein are implants that can be used to repair bone defects and/or tissue surrounding the bone defects.

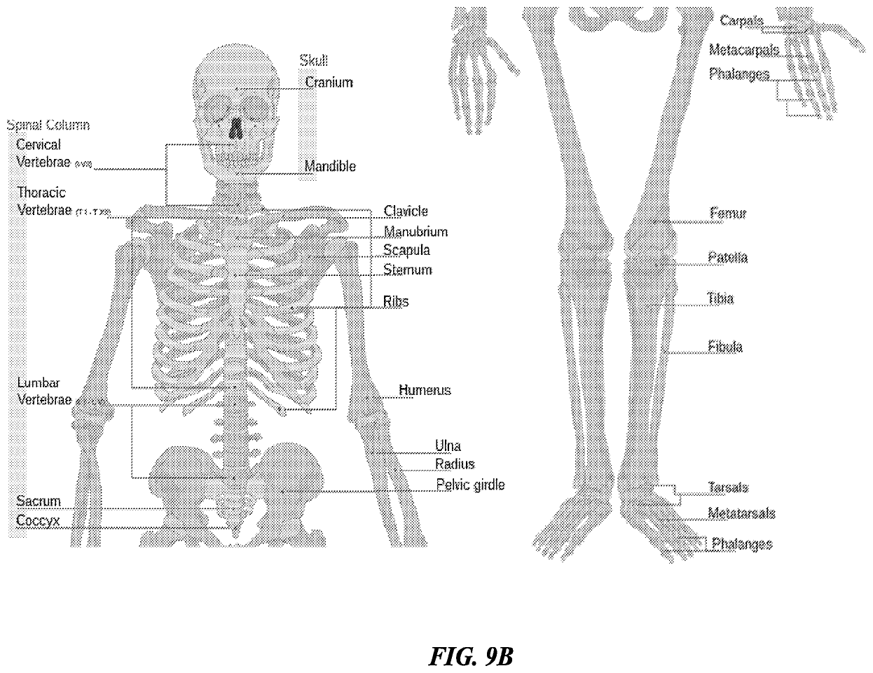

In some embodiments, the implant fills a defect in a cranial bone or a facial bone. FIG. 9A shows examples of cranial bones and facial bones which can have the defect. In some embodiments, the cranial bone comprises an ethmoid bone, a frontal bone, an occipital bone, a parietal bone, a sphenoid bone, or a temporal bone. In some embodiments, the implant fills a defect in a perpendicular plate, a crista galli, an ethmoid bulla, a superior nasal concha, an ethmoidal infundibulum, an orbital plate, a cribriform plate, a superior nasal meatus, a middle nasal concha, an uncinate process, an ethmoidal labyrinth, or any combination thereof of the ethmoid bone. In some embodiments, an implant fills a defect in a squamous part, a nasal part, an orbital plate, a zygomatic plate, or any combination thereof of the frontal bone. In some embodiments, the implant fills a defect in a squamous part, a basilar part, a lateral part, or any combination thereof of the occipital bone. In some embodiments, the implant fills a defect in a body, a greater wing, a lesser wing, a pterygoid process, or any combination thereof of the sphenoid bone. In some embodiments, the implant fills a defect in a squamous part, a mastoid part, a petrous part, a tympanic part, or any combination thereof of the temporal bone.

In some embodiments, the facial bone comprises an inferior nasal conchae, a lacrimal bone, a mandible, a maxillae, a nasal bone, a palatine bone, a vomer, or a zygomatic bone. In some embodiments, the implant fills a defect in a body, a ramus, a coronoid process, a mandibular notch, a condylar process, a head, a neck, a pterygoid fovea, a pterygoid tuberosity, a masseteric tuberosity, an angle, a mandibular foramen, a lingula, a mylohyoid groove, or any combination thereof of the mandible. In some embodiments, the implant fills a defect in a horizontal plate, a perpendicular plate, a pyramidal process, an orbital process, a sphenoidal process, an alveolar bone, or any combination thereof of the palatine bone.

In some embodiments, the maxilla comprises an alveolar bone. In some embodiments, the mandible comprises an alveolar bone. In some embodiments, the alveolar bone comprises one or more sockets for teeth. In some embodiments, the alveolar bone comprises one or more teeth. In some embodiments, the alveolar bone comprises a plurality of teeth. In some embodiments, the alveolar bone comprises at least 1, 2, 3, 4, 5, 6, 7, 8, 9, 10, 11, 12, 13, 14, 15, or 16 teeth. In some embodiments, the alveolar bone comprises at most 1, 2, 3, 4, 5, 6, 7, 8, 9, 10, 11, 12, 13, 14, 15, or 16 teeth. In some embodiments, the alveolar bone comprises 1, 2, 3, 4, 5, 6, 7, 8, 9, 10, 11, 12, 13, 14, 15, or 16 teeth. In some embodiments, a tooth is an upper tooth or a lower tooth. In some embodiments, a tooth is an incisor, a cuspid, a bicuspid, or a molar. In some embodiments, a tooth is a central incisor, a lateral incisor, a cuspid, a first bicuspid, a second bicuspid, a first molar, a second molar, or a third molar.

The implant can fill a defect in bones other than the cranial bone or the facial bone. FIG. 9B shows examples of bones that can have the defect. In some embodiments, the implant fills a defect of a clavicle, a scapula, a sternum, a rib, a vertebra, a cervical vertebrae, a thoracic vertebrae, a lumbar vertebrae, a sacrum, a coccygeal vertebrae, a humerus, a radius, an ulna, a carpal bone, a metacarpal bone, a lunate bone, a triquetral bone, a pisiform bone, a trapezium, a trapezoid bone, a capitate bone, a hamate bone, a phalange, a proximal phalange, an intermediate phalange, a distal phalange, a pelvis, an ilium, a pubis, an ischium, a sacrum, a coccyx, a femur, a patella, a femur, a tibia, a fibula, a tarsal, a metatarsal, a talus, a navicular bone, a calcaneus, a cuboid bone, or a cuneiform bone. In some embodiments, the implant fills a defect of a long bone, a short bone, a flat bone, a round bone, a blunt bone, or an irregular bone.

In some embodiments, the defect comprises a defect formed by a physical trauma, a disease, or a surgery. In some embodiments, the physical trauma comprises a blunt force trauma, a penetrating trauma, or both. In some embodiments, the defect comprises a fracture to any one of the bones disclosed herein. In some embodiments, the fracture comprises a partial fracture, a complete fracture, or both. In some embodiments, the disease comprises osteoporosis, osteopetrosis, osteonecrosis, osteogenesis imperfecta, arthritis, rheumatoid arthritis, fibrous dysplasia, Gorham-Stouth disease, cancer, an infection, a congenital anomaly, or any combination thereof.

The subject can be a mammal. The subject can be a human, chimpanzee, gorilla, dog, cat, rat, cow, horse, donkey, or mouse. The human can be an adult. The human can be a child. The human can be an infant. The human can be from about 1 hour to about 3 years old, about 3 years to about 6 years old, about 6 years to about 12 years old, about 13 years to about 19 years old, about 20 years to about 35 years old, about 35 years to about 45 years old, about 45 years to about 55 years old, about 55 years to about 65 years old, about 65 years to about 75 years old, about 75 years to about 85 years old, or older.

### Attachment Devices

One or more attachment devices can be used, e.g., for fixation of a fractured bone or for osteotomy. The one or more attachment devices can be configured to attach to a native bone, e.g., a fractured bone, of the subject. The one or more attachment devices can be configured to attach to an implant, e.g., an implant configured to fill a defect. The one or more attachment devices can include an adjustment mechanism configured to secure the implant to the subject. The one or more attachment devices can be used to fix an implant and a native bone of the subject in static positions relative to another, in order to improve or restore bone in a defect more reliably and more cohesively. In some cases, at least 1, 2, 3, 4, 5, 6, 7, 8, 9, or 10 attachment devices are used. In some cases less than 10, 9, 8, 7, 6, 5, 4, 3, or 2 attachment devices are used.

The one or more attachment devices can comprise any of a variety of orthopedic devices including, a wire, a pin, a screw, a plate, and an intramedullary nail or rod. The one or more attachment devices can comprise a staple or a clamp. In some embodiments, pins are used to hold pieces of native bone together, or a native bone and an implant together. In some embodiments, pins having a relatively small form factor are used in conditions where bone fragments are too small to be fixed with screws with larger form factors. A pin can be removed after a certain period of time. A pin can comprise a thread of various pitches. Some examples of a pin include Steinman pins, Hagie pins, partially threaded pins, Knowles pins, percutaneous pins, Rush pins, pins with sled-runner tips, or pins with hooked ends. The pin can be used for temporary fixation of the fracture fragments via fracture reduction, or during open surgery. The pin can be used as a guide for the accurate placement of the implant or other attachment devices, e.g., a cannulated screw.

The wire can be used as create a suture or be used as a thread to “sew” an implant to a native bone adjacent to or in proximity to a defect. A wire can be used to “sew” two pieces of native bone fragments. The wire can comprise various diameters. The wire can be smooth or braided. The wire can be used to attach osteotomized bone fragments. The wire can be used to create a tension band that creates a compressive force between an implant and a native bone. Some examples of a wire include a circumferential cerclage wire, Kirschner wire, and a tension band.

The one or more attachment devices can have an adjustment mechanism, including a fixation screw. The adjustment mechanism can be configured to controllably translate in one, two, or three degrees of freedom, and rotate in one, two, or three degrees of freedom to vary a configuration of the bone (e.g., bone fracture) or an implant, in the subject. In some cases, the position or the orientation of the implant can be adjusted using the adjustment mechanism posterior to implanting the implant in a subject's body. The adjustment can be performed with one or more attachment devices that pierce the skin of the subject. This method of adjustment can be useful, when the implant is implanted by cutting through a surface of the subject's skin (e.g., scalp or underneath the chin) that is less visible than a surface of the subject's face. Then, the implant can be adjusted with attachment devices that pierce the subject's skin through small pin holes.

The fixation screw or a bone screw can comprise various types of screws used to pull together an implant and a bone, or two pieces of a bone. The fixation screw can be attached to a plate, a rod, a nail, or any other attachment device that is attached to a bone or an implant. The screw can be used alone to hold an implant in a predetermined position and orientation with respect to a bone, or can be used in combination with a plate, a rod, or a nail. The screw can include a screw head, which can be configured to operably couple with a screwdriver. The screw can include a shank or a core, which can be variable in length and diameter, and can be partially or fully threaded with various pitch. The screw can be self-tapping (e.g., having cutting edges). The screw can include a standard point or a trocar point. The screw can be cortical or cancellous. The cortical screw can be fully threaded. The cortical screw can be configured pierce a bone. The cancellous screw can have deeper threads, larger thread diameters, and larger pitches relative to a cortical screw. The cancellous screw can be used to pierce cancellous bone. The screw can be an interfragmentary screw, which can be configured to cross a fracture or a gap between the implant and a bone. An interfragmentary screw can be used to provide compression between bone fragments and/or an implant to increase stability of a fracture. Other types of screws include compression screws, cortical bone screws, dynamic hip screws, dynamic compression screws, Herbert screws, interfragmentary (lag) screws, interlocking screws, Kurosaka screws, lag screws, malleolar bone screws, syndesmotic screws, cannulated screws, and interference screws.

The one or more attachment devices can have an adjustment mechanism including a plate. The plate can comprise a splint that holds fractured ends of a bone together. The plate can comprise a splint that holds a bone fragment and an implant together. The plate can be extended along the fractured bone and screwed into place and can be removed after healing or left in place. The plate can include a hole through which a screw, a rod, or a nail can be inserted. The number and position of the holes in the plate can be based on an assessment of the stability of an attachment device that can be made through the hole and on the bone or the implant. The plate can be substantially rigid or substantially flexible to disallow or allow small movements or tolerances in the position of the implant. With flexible fixation, the implant can be displaced in relation to a bone when a load is applied on the implant or the bone. The fracture fixation can be considered flexible if it allows appreciable relative displacement of the implant when affixed to a bone under a functional load (e.g., for a mandible bone when chewing). The plate can be a compression plate or a neuralization plate. The compression plate can be used to apply compression forces between the implant and the bone. The compression plate can be dynamically adjusted. Dynamic compression plates can be used to compress a bone and an implant together. The screw in a compression plate can be configured to control a compression force by tightening or loosening the screw on the compression plate to control the magnitude and orientation of the compression force between the implant and the bone that the implant is affixed to. The plate can comprise a neutralization plate. The neutralization plate can comprise a screw to preserve the fixation between the implant and the bone by neutralizing a mechanical stress on the implant, the bone, or both. Other types of plates can be used including reconstruction plates that are notched between the holes which can allow a plate to bend or to be contoured (e.g., to the shape of a natural curvature of a cranial bone or a facial bone), a buttress plate which can support metaphyseal regions, a holt nail plate, a fixed angle nail-plate, a Moe plate, a metallic bone plate, a Morscher plates, an Orion plates, a Sherman plate, a tubular plate, a fracture fixation plate with a circular profile, or a self-compressing plate.

The one or more attachment devices can have an adjustment mechanism including a nail or a rod. The nail or rod can be used to align a bone and an implant. The nail or rod can be inserted through the hollow end or medullary space of a bone that normally contains the marrow. The screw can be used to hold the nail or rod in place and can be inserted in the bone to fix the nail or rod in place and to provide control of rotational and compressive forces. Examples of nails include Enders nails, Gross-Kempfe interlocking nails, Hansen-Street nails, Kuntscher nails (intramedullary nail), Lottes nails, Massie nails (telescoping nail-plate assembly), McGlaughlin nails, Neufeld nails, Sage nails, Sampson nails (hollow cylindrical rod with multiple external flutes), Schneider nails (four-flanged intramedullary nails used for femoral shaft fractures), Smith-Peterson nails (flanged nails for treating fractures of the femoral neck), or triflanged nails (fixation nails used for intracapsular hip fractures). Examples of rods include Knodt rods or reconstruction rods.

The one or more attachment devices can have an adjustment mechanism including a combination of one or more screws, plates, pins, nails, and/or rods. The one or more attachment devices can comprise a non-corrosive material, such as titanium.

In some embodiments, the implant is attached to the bone of the head of the subject without using an attachment device. In some embodiments, the implant is attached to the bone of the head of the subject without using an attachment device using friction. In some embodiments, the implant is attached to the bone of the head of the subject using an adhesive. In some embodiments, the adhesive comprises a biodegradable material. The adhesive can be provided on a portion of the implant that is configured to make contact with the bone of the head of the subject.

### Biologics

In some aspects, the implant provided herein comprises a biologic. In some embodiments, the biologic is provided in the implant prior to implanting. In some embodiments, the biologic is provided in the implant after implanting, for example, by injecting the biologic through a syringe into the implant. In some embodiments, the biologic comprises an osteoinductive material. In some embodiments, the osteoinductive material comprises allograft bone tissue. In some embodiments, the biologic comprises a bone graft material. In some embodiments, the biologic comprises demineralized bone matrix or a bone composite.

The allograft bone can be processed prior to implantation to remove the risk of disease transmission or an immunological response. This processing can remove the osteogenic and osteoinductive properties of the allograft, leaving an osteoconductive scaffold. The osteoconductive scaffold can be morselized.

In some embodiments, the biologic can comprise demineralized bone matrix (DBM). DBM can be prepared by acid extraction of an allograft bone, which can lead to loss of mineralized components but retention of collagen and noncollagenous proteins, including growth factors. DBM can comprise a substrate or a carrier (e.g. glycerol or a polymer). In some embodiments, the demineralized bone matrix comprises demineralized bone matrix fibers and/or demineralized bone matrix chips. In some embodiments, the demineralized bone matrix comprises demineralized bone matrix fibers and demineralized bone matrix chips. In some embodiments, the biologic comprises a bone composite. In some embodiments, the bone composite comprises a bone powder, a polymer, and/or a demineralized bone.

In some embodiments, the demineralized allograft bone material comprises, e.g., a demineralized bone matrix (DBM) comprising fibers, particles, and any combination of thereof. In some embodiments, a bone graft structure comprises a composite bone, which includes a bone powder, a polymer and a demineralized bone. In some embodiments, the biologic comprises a binding agent, which can modify the demineralized allograft bone material or bone graft structure to bind molecules, such as growth factors, cells, cultured cells, or a combination of molecules and cells. In some embodiments, the biologic can comprise BMP-2, rhBMP-2, BMP-4, rhBMP-4, BMP-6, rhBMP-6, BMP-7(OP-1), rhBMP-7, GDF-5, LIM mineralization protein, platelet derived growth factor (PDGF), transforming growth factor-β (TGF-β), insulin-related growth factor-I (IGF-I), insulin-related growth factor-II (IGF-II), fibroblast growth factor (FGF), beta-2-microglobulin (BDGF II), and rhGDF-5.

In some embodiments, the biologic can comprise osteogenic or chondrogenic proteins or peptides, anti-AIDS substances, anti-cancer substances, antibiotics, immunosuppressants, anti-viral substances, enzyme inhibitors, hormones, neurotoxins, opioids, hypnotics, anti-histamines, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and antiadhesion molecules, vasodilating agents, inhibitors of DNA synthesis, inhibitors of RNA synthesis, inhibitors of protein synthesis, anti-hypertensives, analgesics, anti-pyretics, steroidal and non-steroidal anti-inflammatory agents, clonidine, a statin, bone morphogenetic protein, anti-angiogenic factors, angiogenic factors, anti-secretory factors, anticoagulants and/or antithrombotic agents, local anesthetics, prostaglandins, anti-depressants, anti-psychotic substances, anti-emetics, and imaging agents. In some embodiments, the biologic can comprise RNAs, such as siRNA, and osteoclast stimulating factors. In some embodiments, the biologic can be a factor that stops, removes, or reduces the activity of bone growth inhibitors. In some embodiments, the biologic is a growth factor, cytokine, extracellular matrix molecule or a fragment or derivative thereof, for example, a cell attachment sequence such as RGD. In some embodiments the biologic comprises osteogenic or chondrogenic proteins or peptides; DBM powder; collagen, insoluble collagen derivatives, etc., and soluble solids and/or liquids dissolved therein; antimicrobials and/or antibiotics such as erythromycin, bacitracin, neomycin, penicillin, polymycin B, tetracyclines, biomycin, chloromycetin, and streptomycins, cefazolin, ampicillin, azactam, tobramycin, clindamycin and gentamycin, etc.; immunosuppressants; anti-viral substances such as substances effective against hepatitis; enzyme inhibitors; hormones; neurotoxins; opioids; hypnotics; anti-histamines; lubricants; tranquilizers; anti-convulsants; muscle relaxants and anti-Parkinson substances; anti-spasmodics and muscle contractants including channel blockers; miotics and anti-cholinergics; anti-glaucoma compounds; anti-parasite and/or anti-protozoal compounds; modulators of cell-extracellular matrix interactions including cell growth inhibitors and antiadhesion molecules; vasodilating agents; inhibitors of DNA, RNA, or protein synthesis; anti-hypertensives; analgesics; anti-pyretics; steroidal and non-steroidal anti-inflammatory agents; anti-angiogenic factors; angiogenic factors and polymeric carriers containing such factors; anti-secretory factors; anticoagulants and/or antithrombotic agents; local anesthetics; ophthalmics; prostaglandins; anti-depressants; anti-psychotic substances; anti-emetics; imaging agents; biocidal/biostatic sugars such as dextran, glucose, etc.; amino acids; peptides; vitamins; inorganic elements; co-factors for protein synthesis; endocrine tissue or tissue fragments; synthesizers; enzymes such as alkaline phosphatase, collagenase, peptidases, oxidases and the like; polymer cell scaffolds with parenchymal cells; collagen lattices; antigenic agents; cytoskeletal agents; cartilage fragments; living cells such as chondrocytes, bone marrow cells, mesenchymal stem cells; natural extracts; genetically engineered living cells or otherwise modified living cells; expanded or cultured cells; DNA delivered by plasmid, viral vectors, or other member; tissue transplants; autogenous tissues such as blood, serum, soft tissue, bone marrow, or the like; bioadhesives; bone morphogenetic proteins (BMPs including BMP-2); osteoinductive factor (IFO); fibronectin (FN); endothelial cell growth factor (ECGF); vascular endothelial growth factor (VEGF); cementum attachment extracts (CAE); ketanserin; human growth hormone (HGH); animal growth hormones; epidermal growth factor (EGF); interleukins, for example, interleukin-1 (IL-1), interleukin-2 (IL-2); human alpha thrombin; transforming growth factor (TGF-beta); insulin-like growth factors (IGF-1, IGF-2); parathyroid hormone (PTH); platelet derived growth factors (PDGF); fibroblast growth factors (FGF, BFGF, etc.); periodontal ligament chemotactic factor (PDLGF); enamel matrix proteins; growth and differentiation factors (GDF); hedgehog family of proteins; protein receptor molecules; small peptides derived from growth factors above; bone promoters; cytokines; somatotropin; bone digesters; antitumor agents; cellular attractants and attachment agents; immuno-suppressants; permeation enhancers, for example, fatty acid esters such as laureate, myristate and stearate monoesters of polyethylene glycol, enamine derivatives, alpha-keto aldehydes; and nucleic acids.

In some embodiments, the biologic is provided in a composition comprising the biologic. In some embodiments, the composition comprises a polymer. In some embodiments, the polymer is a bioerodible, a bioabsorbable, and/or a biodegradable biopolymer that can provide immediate release or sustained release of other biologics incorporated with the polymer. Some examples of polymers include, but are not limited to, poly(alpha-hydroxy acids), poly(lactide-co-glycolide) (PLGA), polylactide (PLA), polyglycolide (PG), polyethylene glycol (PEG), conjugates of poly(alpha-hydroxy acids), poly(orthoester)s (POE), polyaspirins, polyphosphagenes, collagen, starch, pre-gelatinized starch, hyaluronic acid, chitosans, gelatin, alginates, albumin, fibrin, vitamin E compounds, such as alpha tocopheryl acetate, d-alpha tocopheryl succinate, D,L-lactide, or L-lactide, caprolactone, dextrans, vinylpyrrolidone, polyvinyl alcohol (PVA), PVA-g-PLGA, PEGT-PBT copolymer (polyactive), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate), or combinations thereof.

### Apparatus for Making

In some aspects, provided herein is an apparatus for making an implant of the present disclosure.

The apparatus can comprise a printer configured to build a three-dimensional model by sequential deposition of a plurality of cross-sectional layers. The apparatus can comprise a stage and a depositing mechanism for depositing material on the stage. In some embodiments, the apparatus comprises a computer system (701) configured to control the depositing mechanism and/or the stage (745), as illustrated in FIG. 7.

The apparatus can operate according to a method comprising one or more of the following steps. A flowable material can be deposited on the stage to form a first layer of the implant. A solidification treatment can be applied to the first layer to solidify the first layer. The flowable material can be deposited on the stage to form a second layer of the implant adjacent to the first layer. A solidification treatment can be applied to the second layer to solidify the second layer. The preceding steps can be performed in repetition to form subsequent layers of the implant until the entire implant is made.

In some embodiments, the implant is made to be porous. The flowable material can be deposited in a manner that leaves gaps between the lines of the extrudate. FIGS. 1A-1F show printable models of implants for a mandible. The models can be viewed as a planned trajectory for deposting the extrudate. The trajectory of the extrudate follows various patterns, including zig zags. The trajectories of the extrudate between adjacent layers can criss-cross such to create spaces (e.g., pores) within the implant. Each layer can include one or more continuous extrudate trajectories. An extrudate trajectory can be continuous from one layer to another layer.

In some embodiments, the extrudate trajectories that define the shape of the implant are generated using a computer based on an implant electronic three-dimensional representation. The implant can be modeled with varying degrees of porosity. The computer can autonomously generate the extrudate trajectories based on a user specification (e.g., degree of porosity), device specification (e.g., extrudate thickness), or both. In some embodiments, the layer is substantially planar such that an extrudate trajectory of one layer form a defined plane. In some embodiments, the layer is substantially non-planar such that an extrudate trajectory of one layer does not form a defined plane.

In some embodiments, a material configured to form the implant is flowed through or extruded to form the implant. An extruded material can be solidified through a variety of solidification treatments, including heat treatment, chemical treatment, or both. A solidification treatment can can include sintering, curing, or annealing. Curing can comprise chemical hardening, thermal hardening, vaporization hardening, or any combination thereof.

The apparatus can include a movement mechanism (e.g. a DC motor such as a stepper motor, servo motor or the like) for moving the stage or the extruder. The movement mechanism can be configured to move the stage and/or the extruder such that the relative positions and orientations of the stage and the extruder is manipulatable in one, two, or three translational directions and one, two, or three orientational directions. The movement mechanism can comprise sliders, rails, or other structural elements to allow movement of the stage and/or the extruder.

In some embodiments, the material can comprise poly(alpha-hydroxy acids), poly(lactide-co-glycolide) (PLGA), polylactide (PLA), polyglycolide (PG), polyethylene glycol (PEG), conjugates of poly(alpha-hydroxy acids), poly(orthoester)s (POE), polycaprolactone, polyaspirins, polyphosphagenes, collagen, starch, pre-gelatinized starch, hyaluronic acid, chitosans, gelatin, alginates, albumin, fibrin, vitamin E compounds, such as alpha tocopheryl acetate, d-alpha tocopheryl succinate, D,L-lactide, or L-lactide, caprolactone, dextrans, vinylpyrrolidone, polyvinyl alcohol (PVA), PVA-g-PLGA, PEGT-PBT copolymer (polyactive), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate), or combinations thereof.

In some embodiments, the implant is printed at a point of care (POC). For example, a 3D electronic representation of an implant can be communicated to a 3D printer at a POC location via wired internet, wireless internet, the cloud (730), or any other means disclosed herein suitable for communication digital data. In some embodiments, the implant is printed at a different location, packaged, and shipped to the POC. In some embodiments, the implant is packaged in a hermetically sealed container. In some embodiments, the implant is packaged with one or more attachment devices. The one or more attachment devices can be pre-attached to the implant, or they can be separate from the implant.

### Method of Implanting

In some aspects, the present disclosure provides methods for implanting an implant disclosed herein. In some embodiments, an implant of the present disclosure can be implanted into a head of a subject.

In some embodiments, the methods comprise creating an incision in a skin of a subject adjacent to or in proximity to a defect. A pilot drill can be used to create a pilot hole for an attachment device in a bone adjacent to or in proximity to the defect. A latch reamer can be used to refine or enlarge the pilot hole to a predetermined diameter. The pilot hole can be made to a predetermined depth, which can be measured using a depth gauge. A threaded insert can be placed into the pilot hole, in some cases, with a biologic or an adhesive. In some embodiments, a biologic can be added to the threaded insert. In some embodiments, bone material harvested from drilling and the reaming can be added to the threaded insert. In some embodiments, the implant can be placed within the subject, and attached to the bone of the subject using an attachment device. The attachment device can be a screw that couples the implant to the bone via an extension, a tab, or through the body of the implant.

In some embodiments, the incision wound is closed and sutured before implanting the implant, and the bone can be allowed to heal around the threaded insert for a predetermined duration (e.g., at least 1, 2, 3, 4, 5, 6, 7, 8, 9, or 10 weeks). A new incision can be made in the skin, and the implant can be placed within the subject, and attached to the bone of the subject using an attachment device. The attachment device can be a screw that couples the implant to the bone via an extension, a tab, or through the body of the implant.

In some embodiments, the methods comprise removing a bone from the subject to create a defect. In some cases, bone is removed from a patient when the subject has a bone tumor. For example, in a mandibulectomy, a segment of the mandible bone can be removed. The mandible can be sawed using an oscillating saw to remove the segment. An implant configured to replace the segment can be placed in the subject's mandibular region. The implant can be attached to the remaining portion of the mandible by using one or more of the attachment devices disclosed herein.

In some embodiments, the implanting comprises coupling the implant to the bone of the subject by installing an attachment device through the implant and the bone of the subject. In some embodiments, the attachment device is a screw. In some embodiments, the implant comprises an extension comprising an attachment opening for installing the attachment device.

In some embodiments, the implanting comprises coupling the implant to the bone of the subject by installing a plurality of attachment devices through the implant and the bone of the subject.

### Kits

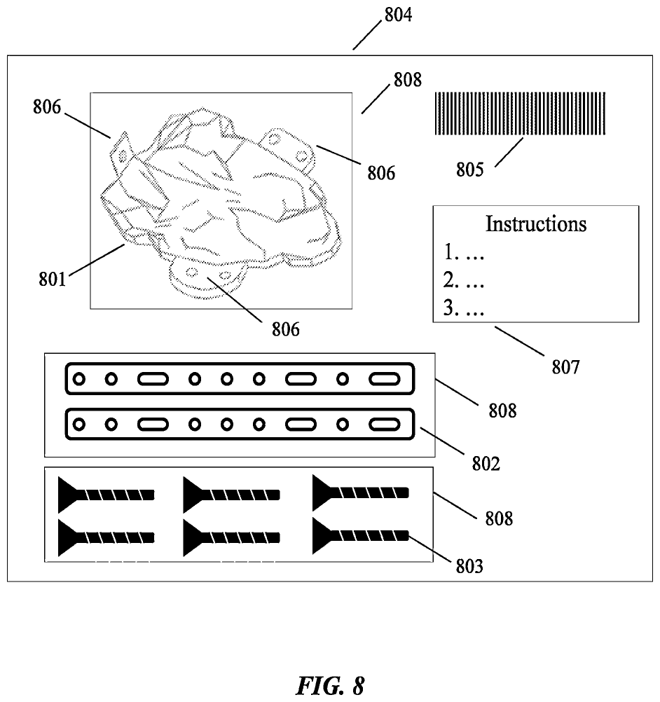

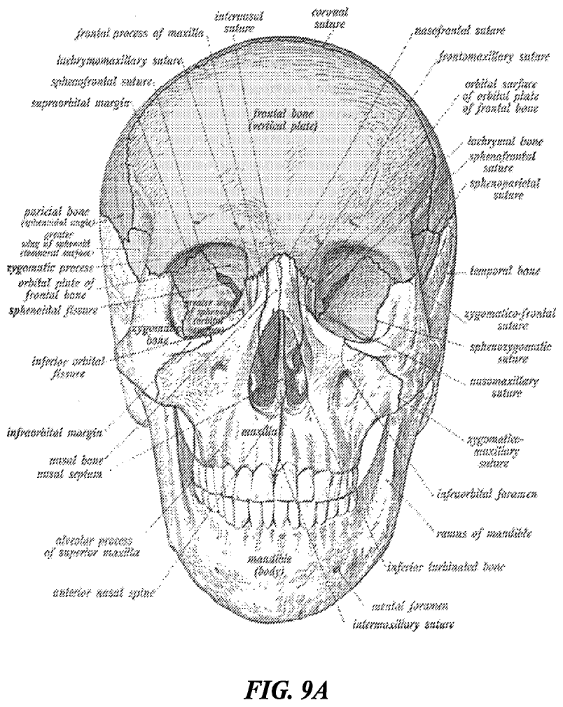

In some aspects, the present disclosure provides a kit comprising an implant (801) of the present disclosure. An example of a kit is shown in FIG. 8. In some embodiments, the kit comprises an attachment device, for example, a plate (802) or a screw (803). In some embodiments, the implant and the attachment device are provided in a hermetically sealed container (804). The kit can comprise packaging material to protect the implant during transport. In some embodiments, the kit comprises an identifier for a subject. In some embodiments, the identifier comprises a barcode or a quick response (QR) code (805). The kit can comprise hermetically sealed containers for the implant or the attachment devices (808).

In some embodiments, the kit comprises a plurality of attachment devices. The plurality of attachment devices can be individually labeled (e.g., using color, alphanumeric codes, shapes, or any combination thereof). The labels can provide visual aids for installing the plurality of attachment devices to the implant, to the bone of a subject, or both.

In some embodiments, the kit comprises a plurality of implants. The plurality of implants can be individually labeled (e.g., using color, alphanumeric codes, shapes, or any combination thereof). The labels can provide visual aids for implanting the implants. The labels can provide orientation or placement information for the implants.

In some embodiments, the kit comprises instructions (807). The instructions can be printed on the container. The instructions can be printed on a sheet. The instructions can be stored on a computer readable medium. The instructions can provide steps for preparing the implant, the attachment device, or both for implanting. The instructions can provide steps for implanting the implant, the attachment device, or both.

In some embodiments, the kit comprises an implant and an attachment device. In some embodiments, the kit comprises an implant and a plate. In some embodiments, the kit comprises an implant and a screw. In some embodiments, the kit comprises an implant and an extension. In some embodiments, the kit comprises an implant and a tab. In some embodiments, the kit comprises an implant and a barcode. In some embodiments, the kit comprises an implant and instructions. In some embodiments, the kit comprises an attachment device and a barcode. In some embodiments, the kit comprises an attachment device and instructions.

### Computer Systems

The present disclosure provides computer systems that are programmed to implement methods of the disclosure. FIG. 7 shows a computer system 701 that is programmed or otherwise configured to, for example, make an implant.

The computer system 701 can regulate various aspects of analysis, calculation, and generation of the present disclosure, such as, for example, making an implant. The computer system 701 can be an electronic device of a user or a computer system that is remotely located with respect to the electronic device. The electronic device can be a mobile electronic device.

The computer system 701 includes a central processing unit (CPU, also “processor” and “computer processor” herein) 705, which can be a single core or multi core processor, or a plurality of processors for parallel processing. The computer system 701 also includes memory or memory location 710 (e.g., random-access memory, read-only memory, flash memory), electronic storage unit 715 (e.g., hard disk), communication interface 720 (e.g., network adapter) for communicating with one or more other systems, and peripheral devices 725, such as cache, other memory, data storage and/or electronic display adapters. The memory 710, storage unit 715, interface 720 and peripheral devices 725 are in communication with the CPU 705 through a communication bus (solid lines), such as a motherboard. The storage unit 715 can be a data storage unit (or data repository) for storing data. The computer system 701 can be operatively coupled to a computer network (“network”) 730 with the aid of the communication interface 720. The network 730 can be the Internet, an internet and/or extranet, or an intranet and/or extranet that is in communication with the Internet.

The network 730 in some cases is a telecommunication and/or data network. The network 730 can include one or more computer servers, which can enable distributed computing, such as cloud computing. For example, one or more computer servers can enable cloud computing over the network 730 (“the cloud”) to perform various aspects of analysis, calculation, and generation of the present disclosure, such as, for example, making an implant. Such cloud computing can be provided by cloud computing platforms such as, for example, Amazon Web Services (AWS), Microsoft Azure, Google Cloud Platform, and IBM cloud. The network 730, in some cases with the aid of the computer system 701, can implement a peer-to-peer network, which can enable devices coupled to the computer system 701 to behave as a client or a server.

The CPU 705 can comprise one or more computer processors and/or one or more graphics processing units (GPUs). The CPU 705 can execute a sequence of machine-readable instructions, which can be embodied in a program or software. The instructions can be stored in a memory location, such as the memory 710. The instructions can be directed to the CPU 705, which can subsequently program or otherwise configure the CPU 705 to implement methods of the present disclosure. Examples of operations performed by the CPU 705 can include fetch, decode, execute, and writeback.

The CPU 705 can be part of a circuit, such as an integrated circuit. One or more other components of the system 701 can be included in the circuit. In some cases, the circuit is an application specific integrated circuit (ASIC).

The storage unit 715 can store files, such as drivers, libraries and saved programs. The storage unit 715 can store user data, e.g., user preferences and user programs. The computer system 701 in some cases can include one or more additional data storage units that are external to the computer system 701, such as located on a remote server that is in communication with the computer system 701 through an intranet or the Internet.

The computer system 701 can communicate with one or more remote computer systems through the network 730. For instance, the computer system 701 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 701 via the network 730.

Methods as described herein can be implemented by way of machine (e.g., computer processor) executable code stored on an electronic storage location of the computer system 701, such as, for example, on the memory 710 or electronic storage unit 715. The machine executable or machine readable code can be provided in the form of software. During use, the code can be executed by the processor 705. In some cases, the code can be retrieved from the storage unit 715 and stored on the memory 710 for ready access by the processor 705. In some situations, the electronic storage unit 715 can be precluded, and machine-executable instructions are stored on memory 710.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 701, can be embodied in programming. Various aspects of the technology can be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which can provide non-transitory storage at any time for the software programming. All or portions of the software can at times be communicated through the Internet or various other telecommunication networks. Such communications, for example, can enable loading of the software from one computer or processor into another, for example, from a management server or host computer into the computer platform of an application server. Thus, another type of media that can bear the software elements includes optical, electrical and electromagnetic waves, such as used across physical interfaces between local devices, through wired and optical landline networks and over various air-links. The physical elements that carry such waves, such as wired or wireless links, optical links or the like, also can be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, can take many forms, including but not limited to, a tangible storage medium, a carrier wave medium or physical transmission medium. Non-volatile storage media include, for example, optical or magnetic disks, such as any of the storage devices in any computer(s) or the like, such as can be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media can take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media can be involved in carrying one or more sequences of one or more instructions to a processor for execution.

The computer system 701 can include or be in communication with an electronic display 735 that comprises a user interface (UI) 740 for providing, for example, making an implant. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 705. The algorithm can, for example, be used for making an implant.

### List of Embodiments

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.
Embodiment 1. An implant, comprising: (a) a porous body comprising a form that fills a defect of a bone of a head of a subject; (b) a recess in the porous body, wherein the recess comprises a dimension larger than an average pore dimension of the porous body; and (c) a biologic disposed in the recess.
Embodiment 2. The implant of Embodiment 1, wherein the biologic comprises a biodegradable ceramic.
Embodiment 3. The implant of Embodiment 2, wherein the biodegradable ceramic is beta-tricalcium phosphate (beta-TCP).
Embodiment 4. The implant of any one of Embodiments 1-3, wherein the biologic comprises demineralized bone matrix, morselized bone, bone marrow aspirate, bone morphogenetic protein 2 (BMP2), beta-TCP, or any combination thereof.
Embodiment 5. The implant of any one of Embodiments 1-4, wherein two most distant points on a surface of the recess are separated by at least 7 millimeters.
Embodiment 6. The implant of any one of Embodiments 1-5, wherein two nearest points on a surface of the recess are separated by at most 2 millimeters.
Embodiment 7. The implant of any one of Embodiments 1-6, wherein the recess comprises a curved surface.
Embodiment 8. The implant of any one of Embodiments 1-7, wherein the recess forms a channel from one end of the porous body to another end of the porous body.
Embodiment 9. The implant of any one of Embodiments 1-8, wherein the recess comprises an opening that opens inwards to the subject.
Embodiment 10. The implant of any one of Embodiments 1-9, wherein the recess comprises a plurality of recesses.
Embodiment 11. The implant of Embodiment 10, wherein each of the plurality of recesses comprises different dimensions.
Embodiment 12. The implant of any one of Embodiments 1-11, wherein the bone is a mandible of the head of the subject.
Embodiment 13. The implant of any one of Embodiments 1-11, wherein the bone is a maxilla of the head of the subject.
Embodiment 14. The implant of any one of Embodiments 1-11, wherein the bone is a nasal bone of the head of the subject.
Embodiment 15. The implant of any one of Embodiments 1-11, wherein the bone is an orbital bone of the head of the subject.
Embodiment 16. The implant of any one of Embodiments 1-11, wherein the bone is a cranial bone of the head of the subject.
Embodiment 17. The implant of Embodiment 16, wherein the porous body comprises a chevron hole.
Embodiment 18. The implant of any one of Embodiments 1-17, wherein the porous body comprises a porosity of at least 25, 30, 35, 40, 45, 50, 55, 60, or 65%.
Embodiment 19. An implant, comprising: (a) a porous body comprising a form that fills a defect of a bone of a head of a subject; (b) a biologic disposed in the porous body; and (c) an extension connected to the porous body, wherein the extension is configured to lay against the bone of the head of the subject.
Embodiment 20. The implant of Embodiment 19, wherein the bone is a mandible of the head of the subject.
Embodiment 21. The implant of Embodiment 20, wherein the extension is configured to lay at least 2 mm above a mandibular division of a trigeminal nerve of the head of the subject.
Embodiment 22. The implant of any one of Embodiments 19-21, wherein the bone is an orbital bone of the head of the subject.
Embodiment 23. The implant of Embodiment 22, wherein the extension is configured to lay against a frontal bone of the orbital bone.
Embodiment 24. The implant of Embodiment 22 or 23, wherein the extension is configured to lay against a maxilla of the orbital bone.
Embodiment 25. The implant of any one of Embodiments 22-24, wherein the extension is configured to lay against a lacrimal bone of the orbital bone.
Embodiment 26. The implant of any one of Embodiments 19-21, wherein the bone is a maxilla of the head of the subject.
Embodiment 27. The implant of any one of Embodiments 19-21, wherein the bone is a nasal bone of the head of the subject.
Embodiment 28. The implant of any one of Embodiments 19-21, wherein the bone is a cranial bone of the head of the subject.
Embodiment 29. The implant of any one of Embodiments 19-28, wherein the extension comprises extends from the porous body by at least 2 millimeters.
Embodiment 30. The implant of any one of Embodiments 19-29, wherein the extension comprises a thickness of at least about 0.8 millimeters.
Embodiment 31. The implant of any one of Embodiments 19-30, wherein the extension is substantially parallel to a surface of the porous body.
Embodiment 32. The implant of any one of Embodiments 19-31, wherein the extension is at an angle to a surface of the porous body.
Embodiment 33. The implant of Embodiment 32, wherein the angle is at least 5 degrees.
Embodiment 34. The implant of any one of Embodiments 19-33, wherein the extension comprises an attachment opening.
Embodiment 35. The implant of Embodiment 34, wherein the extension comprises a plurality of attachment openings.
Embodiment 36. The implant of Embodiment 35, wherein the plurality of attachment openings are oriented at an angle with respect to another.
Embodiment 37. The implant of any one of Embodiments 34-36, wherein the attachment opening is pitched.
Embodiment 38. The implant of any one of Embodiments 19-37, wherein the extension comprises a rounded edge.
Embodiment 39. The implant of any one of Embodiments 19-38, wherein the implant comprises a plurality of extensions.
Embodiment 40. The implant of any one of Embodiments 19-39, wherein the biologic comprises a biodegradable ceramic.
Embodiment 41. The implant of Embodiment 40, wherein the biodegradable ceramic is beta-TCP.
Embodiment 42. The implant of any one of Embodiments 19-41, wherein the biologic comprises demineralized bone matrix, morselized bone, bone marrow aspirate, BMP2, or any combination thereof.
Embodiment 43. The implant of any one of Embodiments 19-42, wherein the porous body comprises a porosity of at least 25, 30, 35, 40, 45, 50, 55, 60, or 65%.
Embodiment 44. An implant, comprising: (a) a porous body comprising a form that fills a defect of a bone of a head of a subject, wherein the porous body comprises a biodegradable synthetic polymer; and (b) an extension connected to the porous body, wherein the extension is configured to lay against the bone of the subject.
Embodiment 45. The implant of Embodiment 44, wherein the biodegradable synthetic polymer is polycaprolactone.
Embodiment 46. The implant of Embodiment 44 or 45, wherein the bone is a mandible of the head of the subject.
Embodiment 47. The implant of Embodiment 46, wherein the extension is configured to lay at least 2 mm above a mandibular division of a trigeminal nerve of the head of the subject.
Embodiment 48. The implant of Embodiment 44 or 45, wherein the bone is an orbital bone of the head of the subject.
Embodiment 49. The implant of Embodiment 48, wherein the extension is configured to lay against a frontal bone of the orbital bone.
Embodiment 50. The implant of Embodiment 48 or 49, wherein the extension is configured to lay against a maxilla of the orbital bone.
Embodiment 51. The implant of any one of Embodiments 48-50, wherein the extension is configured to lay against a lacrimal bone of the orbital bone.
Embodiment 52. The implant of Embodiment 44 or 45, wherein the bone is a maxilla of the head of the subject.
Embodiment 53. The implant of Embodiment 44 or 45, wherein the bone is a nasal bone of the head of the subject.
Embodiment 54. The implant of Embodiment 44 or 45, wherein the bone is a cranial bone of the head of the subject.
Embodiment 55. The implant of any one of Embodiments 44-54, wherein the extension comprises extends from the porous body by at least 2 millimeters.
Embodiment 56. The implant of any one of Embodiments 44-55, wherein the extension comprises a thickness of at least about 0.8 millimeters.
Embodiment 57. The implant of any one of Embodiments 44-56, wherein the extension is substantially parallel to a surface of the porous body.
Embodiment 58. The implant of any one of Embodiments 44-56, wherein the extension is at an angle to a surface of the porous body.
Embodiment 59. The implant of any one of Embodiments 44-58, wherein the extension comprises an attachment opening.
Embodiment 60. The implant of Embodiment 59, wherein the extension comprises a plurality of attachment openings.
Embodiment 61. The implant of Embodiment 60, wherein the attachment opening is pitched.
Embodiment 62. The implant of any one of Embodiments 44-61, wherein the implant comprises a plurality of extensions.
Embodiment 63. The implant of any one of Embodiments 44-62, wherein the biologic comprises a biodegradable ceramic.
Embodiment 64. The implant of Embodiment 63, wherein the biodegradable ceramic is beta-TCP.
Embodiment 65. The implant of any one of Embodiments 44-64, wherein the biologic comprises demineralized bone matrix, morselized bone, bone marrow aspirate, BMP2, or any combination thereof.
Embodiment 66. The implant of any one of Embodiments 44-65, wherein the porous body comprises a porosity of at least 25, 30, 35, 40, 45, 50, 55, 60, or 65%.
Embodiment 67. A kit comprising the implant of any one of Embodiments 1-66.
Embodiment 68. A method comprising implanting the implant of any one of Embodiments 1-66 into the head of the subject.
Embodiment 69. The method of Embodiment 68, wherein the implanting comprises coupling the implant to the bone of the subject by installing an attachment device through the implant and the bone of the subject.
Embodiment 70. The method of any one of Embodiments 68-69, wherein the attachment device is a screw.
Embodiment 71. The method of any one of Embodiments 68-70, wherein the implant comprises an extension comprising an attachment opening for installing the attachment device.
Embodiment 72. The method of any one of Embodiments 68-71, wherein the attachment device comprises a plurality of attachment devices.
Embodiment 73. A method of implanting an implant, comprising: (a) providing the implant comprising a porous body that fills a defect of a bone of a head of a subject, wherein the porous body comprises a biologic; and (b) coupling the implant to the bone of the subject by installing an attachment device through the implant and the bone of the subject, wherein the attachment device comprises a non-corrosive material.
Embodiment 74. The method of Embodiment 73, wherein the biologic comprises a biodegradable ceramic.
Embodiment 75. The method of Embodiment 74, wherein the biodegradable ceramic is beta-TCP.
Embodiment 76. The method of Embodiment 75, wherein the biologic comprises demineralized bone matrix, morselized bone, bone marrow aspirate, BMP2, or any combination thereof.
Embodiment 77. The method of any one of Embodiments 73-76, wherein the non-corrosive material comprises a non-corrosive metal.
Embodiment 78. The method of Embodiment 77, wherein the non-corrosive metal is titanium.
Embodiment 79. The method of any one of Embodiments 73-78, wherein the bone is a mandible of the head of the subject.
Embodiment 80. The method of any one of Embodiments 73-78, wherein the bone is a maxilla of the head of the subject.
Embodiment 81. The method of any one of Embodiments 73-78, wherein the bone is a nasal bone of the head of the subject.
Embodiment 82. The method of any one of Embodiments 73-78, wherein the bone is an orbital bone of the head of the subject.
Embodiment 83. The method of any one of Embodiments 73-78, wherein the bone is a cranial bone of the head of the subject.
Embodiment 84. A method of implanting an implant, comprising: (a) providing the implant comprising a porous body that fills a defect of a bone of a head of a subject, wherein the porous body comprises a biodegradable synthetic polymer; and (b) coupling the implant to the bone of the subject by installing an attachment device through the implant and the bone of the subject, wherein the attachment device comprises a non-corrosive material.
Embodiment 85. The method of Embodiment 84, wherein the biodegradable synthetic polymer is polycaprolactone.
Embodiment 86. The method of Embodiment 84 or 85, wherein the non-corrosive material comprises a non-corrosive metal.
Embodiment 87. The method of Embodiment 86, wherein the non-corrosive metal is titanium.
Embodiment 88. The method of any one of Embodiments 84-87, wherein the implant comprises an extension comprising an attachment opening for installing the attachment device.
Embodiment 89. The method of Embodiment 88, wherein the attachment opening is pitched.
Embodiment 90. The method of Embodiment 89, wherein the attachment device is a screw.
Embodiment 91. The method of any one of Embodiments 84-90, further comprising coupling the implant to the bone of the subject by installing a plurality of attachment devices through the implant and the bone of the subject.
Embodiment 92. The method of any one of Embodiments 84-91, wherein the bone is a mandible of the head of the subject.
Embodiment 93. The method of any one of Embodiments 84-91, wherein the bone is a maxilla of the head of the subject.
Embodiment 94. The method of any one of Embodiments 84-91, wherein the bone is a nasal bone of the head of the subject.
Embodiment 95. The method of any one of Embodiments 84-91, wherein the bone is an orbital bone of the head of the subject.
Embodiment 96. The method of any one of Embodiments 84-91, wherein the bone is a cranial bone of the head of the subject.
Embodiment 97. A method of implanting an implant, comprising: (a) providing the implant comprising a porous body that fills a defect of a bone of a head of a subject, wherein the porous body comprises a porosity of at least 25%; and (b) coupling the implant to the bone of the subject by installing an attachment device through the implant and the bone of the subject, wherein the attachment device comprises a non-corrosive material.
Embodiment 98. The method of Embodiment 97, wherein the porosity is at least 30, 35, 40, 45, 50, 55, 60, or 65%.
Embodiment 99. The method of Embodiment 97 or 98, wherein the non-corrosive material comprises a non-corrosive metal.
Embodiment 100. The method of Embodiment 99, wherein the non-corrosive metal is titanium.
Embodiment 101. The method of any one of Embodiments 97-100, wherein the implant comprises an extension comprising an attachment opening for installing the attachment device.
Embodiment 102. The method of Embodiment 101, wherein the attachment opening is pitched.
Embodiment 103. The method of Embodiment 102, wherein the attachment device is a screw.
Embodiment 104. The method of any one of Embodiments 97-100, wherein the attachment device is a plate.
Embodiment 105. The method of any one of Embodiments 97-104, further comprising coupling the implant to the bone of the subject by installing a plurality of attachment devices through the implant and the bone of the subject.
Embodiment 106. The method of any one of Embodiments 97-105, wherein the bone is a mandible of the head of the subject.
Embodiment 107. The method of any one of Embodiments 97-105, wherein the bone is a maxilla of the head of the subject.
Embodiment 108. The method of any one of Embodiments 97-105, wherein the bone is a nasal bone of the head of the subject.
Embodiment 109. The method of any one of Embodiments 97-105, wherein the bone is an orbital bone of the head of the subject.
Embodiment 110. The method of any one of Embodiments 97-105, wherein the bone is a cranial bone of the head of the subject.
Embodiment 111. A kit comprising: (a) an implant comprising a porous body that fills a defect of a bone of a head of a subject, wherein the porous body comprises a biologic; and (b) an attachment device comprising a non-corrosive material.
Embodiment 112. The kit of Embodiment 111, wherein the biologic comprises a biodegradable ceramic.
Embodiment 113. The kit of Embodiment 112, wherein the biodegradable ceramic is beta-TCP.
Embodiment 114. The kit of Embodiment 113, wherein the biologic comprises demineralized bone matrix, morselized bone, bone marrow aspirate, BMP2, or any combination thereof.
Embodiment 115. The kit of any one of Embodiments 111-114, wherein the non-corrosive material comprises a non-corrosive metal.
Embodiment 116. The kit of Embodiment 115, wherein the non-corrosive metal is titanium.
Embodiment 117. The kit of any one of Embodiments 111-116, wherein the bone is a mandible of the head of the subject.
Embodiment 118. The kit of any one of Embodiments 111-116, wherein the bone is a maxilla of the head of the subject.
Embodiment 119. The kit of any one of Embodiments 111-116, wherein the bone is a nasal bone of the head of the subject.
Embodiment 120. The kit of any one of Embodiments 111-116, wherein the bone is an orbital bone of the head of the subject.
Embodiment 121. The kit of any one of Embodiments 111-116, wherein the bone is a cranial bone of the head of the subject.
Embodiment 122. The kit of any one of Embodiments 111-121, wherein the implant and the attachment device are provided in a hermetically sealed container.
Embodiment 123. The kit of any one of Embodiments 111-122, further comprising an identifier for the subject.
Embodiment 124. The kit of any one of Embodiments 111-123, wherein the identifier comprise a barcode or a quick response (QR) code.
Embodiment 125. A kit comprising: (a) an implant comprising a porous body that fills a defect of a bone of a head of a subject, wherein the porous body comprises a biodegradable synthetic polymer; and (b) an attachment device through the implant and the bone of the subject, wherein the attachment device comprises a non-corrosive material.
Embodiment 126. The kit of Embodiment 125, wherein the biodegradable synthetic polymer is polycaprolactone.
Embodiment 127. The kit of Embodiment 125 or 126, wherein the non-corrosive material comprises a non-corrosive metal.
Embodiment 128. The kit of Embodiment 127, wherein the non-corrosive metal is titanium.
Embodiment 129. The kit of any one of Embodiments 125-128, wherein the implant comprises an extension comprising an attachment opening for installing the attachment device.
Embodiment 130. The kit of Embodiment 129, wherein the attachment opening is pitched.
Embodiment 131. The kit of Embodiment 130, wherein the attachment device is a screw.
Embodiment 132. The kit of any one of Embodiments 125-131, further comprising a plurality of attachment devices.
Embodiment 133. The kit of any one of Embodiments 125-132, wherein the bone is a mandible of the head of the subject.
Embodiment 134. The kit of any one of Embodiments 125-132, wherein the bone is a maxilla of the head of the subject.
Embodiment 135. The kit of any one of Embodiments 125-132, wherein the bone is a nasal bone of the head of the subject.
Embodiment 136. The kit of any one of Embodiments 125-132, wherein the bone is an orbital bone of the head of the subject.
Embodiment 137. The kit of any one of Embodiments 125-132, wherein the bone is a cranial bone of the head of the subject.
Embodiment 138. The kit of any one of Embodiments 125-137, wherein the implant and the attachment device are provided in a hermetically sealed container.
Embodiment 139. The kit of any one of Embodiments 125-138, further comprising an identifier for the subject.
Embodiment 140. The kit of any one of Embodiments 125-139, wherein the identifier comprise a barcode or a quick response (QR) code.
Embodiment 141. A kit comprising: (a) an implant comprising a porous body that fills a defect of a bone of a head of a subject, wherein the porous body comprises a porosity of at least 25%; and (b) an attachment device through the implant and the bone of the subject, wherein the attachment device comprises a non-corrosive material.
Embodiment 142. The kit of Embodiment 141, wherein the porosity is at least 30, 35, 40, 45, 50, 55, 60, or 65%.
Embodiment 143. The kit of Embodiment 141 or 142, wherein the non-corrosive material comprises a non-corrosive metal.
Embodiment 144. The kit of Embodiment 143, wherein the non-corrosive metal is titanium.
Embodiment 145. The kit of any one of Embodiments 141-144, wherein the implant comprises an extension comprising an attachment opening for installing the attachment device.
Embodiment 146. The kit of Embodiment 145, wherein the attachment opening is pitched.
Embodiment 147. The kit of Embodiment 146, wherein the attachment device is a screw.
Embodiment 148. The kit of any one of Embodiments 141-147, wherein the attachment device is a plate.
Embodiment 149. The kit of any one of Embodiments 141-148, further comprising a plurality of attachment devices.
Embodiment 150. The kit of any one of Embodiments 141-149, wherein the bone is a mandible of the head of the subject.
Embodiment 151. The kit of any one of Embodiments 141-149, wherein the bone is a maxilla of the head of the subject.
Embodiment 152. The kit of any one of Embodiments 141-149, wherein the bone is a nasal bone of the head of the subject.
Embodiment 153. The kit of any one of Embodiments 141-149, wherein the bone is an orbital bone of the head of the subject.
Embodiment 154. The kit of any one of Embodiments 141-149, wherein the bone is a cranial bone of the head of the subject.
Embodiment 155. The kit of any one of Embodiments 141-154, wherein the implant and the attachment device are provided in a hermetically sealed container.
Embodiment 156. The kit of any one of Embodiments 141-155, further comprising an identifier for the subject.
Embodiment 157. The kit of any one of Embodiments 141-156, wherein the identifier comprise a barcode or a quick response (QR) code.
Embodiment 158. A method of making an implant, comprising: (a) obtaining an electronic three-dimensional representation of a bone of a subject; (b) removing noise from the electronic three-dimensional representation of the bone of the subject to generate a denoised electronic three-dimensional representation; (c) creating an implant electronic three-dimensional representation that complements the denoised electronic three-dimensional representation; and (d) using the implant electronic three-dimensional representation to generate the implant.
Embodiment 159. The method of Embodiment 158, further comprising, after (c), electronically designing an attachment opening, an extension, or a recess in the implant electronic three-dimensional representation.
Embodiment 160. The method of Embodiment 158 or 159, wherein a position of the attachment opening, the extension, or the recess is selected based on: a thickness of the bone, a fragility of the bone, an absence of a nerve in the subject proximal to the position, an expected torque at a location proximal to the position during installation of an attachment at the position, or any combination thereof.
Embodiment 161. The method of any one of Embodiments 158-160, wherein the implant is generated using 3-dimensional (3-D) printing.
Embodiment 162. The method of Embodiment 161, wherein the 3-D printing comprises layer-by-layer printing.
Embodiment 163. The method of Embodiment 162, wherein a longitudinal axis of the attachment opening is substantially parallel to a normal of a plane of a layer in the implant printed by the layer-by-layer printing.
Embodiment 164. The method of Embodiment 163, wherein an angle between the longitudinal axis and the normal is less than about 45, 30, 15, 10, 5, 4, 3, 2, or 1 degrees.
Embodiment 165. The method of any one of Embodiments 158-164, wherein the using the implant electronic three-dimensional representation to generate the implant is based on a defect of the bone of the subject.
Embodiment 166. The method of any one of Embodiments 158-164, wherein the generated implant fills in a defect or a void of the bone of the subject.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art can alternatively be used.

### Example 1: Making and Installing an Implant

A patient with a mandibular cancer visits a surgeon. A 3-D electronic representation of the patient's mandible is obtained using a suitable instrument, for example X-Ray, magnetic resonance imaging (MRI), positron emission tomography (PET), or computed tomography (CT). A segment of the mandible is identified to have a malignant tumor or lesion. The surgeon prepares a surgical plan to remove the segment and replace the segment with an implant.

The surgical plan is based on a 3-D electronic representation of the mandible where the segment is removed to create a defect. The 3-D electronic representation is sent to an implant designer, who uses the 3-D electronic representation creating an implant electronic three-dimensional representation that complements the defect. The 3-D electronic representation can be processed by removing noise.

The 3-D electronic representation of the implant is provided to the surgeon, who positions one or more attachment openings, extensions, or recesses in the implant. The positions of the attachment openings, the extensions, and the recesses can be based on: a thickness of the bone, a fragility of the bone, an absence of a nerve in the subject proximal to the position, an expected torque at a location proximal to the position during installation of an attachment device at the position, or any combination thereof. The surgeon can place the attachment openings and the extensions to provide optimum stability and security for the implant during the healing process. The surgeon can place the recesses to provide biologics within the recesses, which can accelerate the healing process or to reduce possibility of infection.

The 3-D electronic representation of the implant with the one or more attachment openings, extensions, or recesses is used to 3-D print the implant. The 3-D printing can comprise layer-by-layer printing. The longitudinal axis of the attachment opening can be substantially parallel to a normal of a plane of a layer in the implant printed by the layer-by-layer printing. This relative orientation between the attachment opening and the layer of the implant can reduce the probability of the implant cracking or fragmenting when an attachment device is installed through the attachment opening. The extensions can be coupled to the implant after 3-D printing.

A kit is provided to the surgeon with the implant and appropriate attachment devices for installing the implant in the patient. The surgeon can begin the surgery by performing a neck dissection to gain access to the mandible. The surgeon can saw the mandible using an oscillating saw to remove the segment with the malignant tumor or lesion, creating a defect in the mandible. The implant can be placed in the defect and attached to the remaining mandible according to the surgical plan.

After placement of the implant in the defect, the open wound can be closed. Over a period of, e.g., months, the implant can gradually degrade and native bone tissue can grow to replace the implant. In some cases, the implant can completely degrade as the implant is replaced with natural bone. After sufficient time has passed, there may be no part of the implant that remains permanently. The degradation rate of the implant in the body can depend on biological factors of the subject. The degradation rate of the implant can depend on the size of the scaffold can contribute to slight deviations in the rate at which the scaffold degrades but the rate should be relatively consistent.

### Example 2: Implant for a Mandible Bone Defect

An electronic representation of an implant (“the implant”) was created for use as a mandible bone regeneration scaffold, as shown in FIGS. 2A-2B. The implant is configured to replace a loss of natural bone along the back of a mandible of a subject. The implant is configured to rest on top of native bone of the subject with a height of approximately 5 mm and about 2 mm above the mandibular division of the trigeminal nerve. The height of 5 mm is useful for matching the height of native bone missing in the defect. The implant can be configured to rest about 2 mm above the trigeminal nerve, which can provide surgeons with adequate space to avoid striking the trigeminal nerve when fixating the scaffold.

The implant has an overhang with two screw holes having diameters of about 0.8 mm. The dimensions of the overhang (e.g., thickness, length) and the number, the positions, and the diameters of the screw holes can be different based on case-specific needs of the subject or the surgeon.

The implant has a recess created in a length-wise direction which is configured to hold or be filled with biologics. The biologics can be any one of demineralized bone matrix, morselized bone, bone marrow aspirate, BMP2, or other biologics. The recess can extend the full length or a partial length of the implant. Multiple recesses can be provided, and the dimensions of each recess can be different from one another.

The implant is made of a biodegradable thermoplastic. The implant can be made of a biodegradable thermoset. The implant can have a porosity of approximately 25% to 65%. The implant can be printed with a 3D printer nozzle size of about 0.4 mm. The 3D printer can print the implant with a layer thickness ranging from 0.1 to 0.4 mm.

### Example 3: Implant for an Orbital Bone Defect

An electronic representation of an implant (“the implant”) was created for use as an orbital bone regeneration scaffold, as shown in FIGS. 3A-3D. The implant is configured to replace fractured bone at the base of the left orbital. The implant is configured to replicate the natural curvature of the eye socket. The implant is configured to meet the native bone near the defect. Variations of the implant designs are shown in FIGS. 3B-3D.

FIG. 3B shows a first design comprising an extension tab towards the front of the eye socket. The extension tab can provide easier surgical accessibility when placed towards the front of the eye socket. The extension tab has two screw holes for allowing fixation to native bone. Fixing the implant can improve stability of the implant while providing manageable or reduced disturbance to the natural movement or appearance of the subject.

FIG. 3C shows an implant with an extension tab that is placed towards maxilla and lacrimal bone. This implant has one screw hole, which can be used for fixing the implant to native bone while providing reduced disturbance to the natural movement or appearance of the subject. FIG. 3D shows an implant with an extension tab placed towards the back of the eye socket. The extension tab placed towards the back of the eye socket or towards the maxilla and lacrimal bone provide reduced disturbance to the appearance of the subject.

The dimensions of the extension tab (e.g., thickness, length) and the number, the positions, and the diameters of the screw holes can be different based on case-specific needs of the subject or the surgeon.

The implant is made of a biodegradable thermoplastic. The implant can have a porosity of approximately 25% to 65%. The implant can be printed with a 3D printer nozzle size of about 0.4 mm. The 3D printer can print the implant with a layer thickness ranging from 0.1 to 0.4 mm.

### Example 4: Implant for a Maxilla and Nasal Bone Defect

An electronic representation of an implant (“the implant”) was created for use as a maxilla and nasal bone regeneration scaffold, as shown in FIGS. 4A-4E. The implant is configured to fit snuggly in a defect that spanned from the maxilla to the nasal bone. The implant is made of a biodegradable thermoplastic. The implant can have a porosity of approximately 25% to 65%. The implant can be printed with a 3D printer nozzle size of about 0.4 mm. The 3D printer can print the implant with a layer thickness ranging from 0.1 to 0.4 mm. The locations and dimensions of the overhang, screw holes, and pockets can be adjusted based on case-specific needs of the subject or the surgeon.

### Example 5: Implant for a Cranial Bone Defect

An electronic representation of an implant (“the implant”) was created for use as a cranial bone regeneration scaffold, as shown in FIGS. 5A-5C. The implant is configured to replace a section of bone located toward the center of the frontal region of the skull. To match the natural curvature of the skull, the shape of the implant was designed using remaining bone in the frontal region of the skull as outline. FIGS. 5B-5C show two different designs.

FIG. 5B shows a design with multiple chevron-shaped holes (501). The chevron-shaped holes are configured to relieve pressure when implanted.

FIG. 5C shows a design with reduced number of chevron holes, with respect to the design of FIG. 5B. The design also has three sets (sets of two) pilot holes (502) for placing screws that are configured to attach the scaffold to titanium plates.

The implant can have a porosity of approximately 25% to 65%. The implant can be printed with a 3D printer nozzle size of about 0.4 mm. The 3D printer can print the implant with a layer thickness ranging from 0.1 to 0.4 mm. The locations and dimensions of the overhang, screw holes, and pockets can be adjusted based on case-specific needs of the subject or the surgeon.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure can be employed in practicing the present disclosure. It is intended that the following claims define the scope of the present disclosure and that methods and structures within the scope of these claims and their equivalents be covered thereby.

What is claimed is:

1. A method of implanting an implant, comprising:
(a) providing the implant comprising a porous body that fills a defect of a bone of a head of a subject, wherein the porous body comprises a biologic; and
(b) coupling the implant to the bone of the subject by installing an attachment device through the implant and the bone of the subject, wherein the attachment device comprises a non-corrosive material.

2. The method of claim 1, wherein the biologic comprises a biodegradable ceramic.

3. The method of claim 1, wherein the biologic comprises demineralized bone matrix, morselized bone, bone marrow aspirate, BMP2, or any combination thereof.

4. The method of claim 1, wherein the non-corrosive material comprises a non-corrosive metal.

5. The method of claim 1, wherein the implant comprises an extension comprising an attachment opening for installing the attachment device.

6. The method of claim 1, wherein porous body comprises a recess, and wherein the biologic is disposed in the recess.

7. An implant, comprising:
(a) a porous body comprising a form that fills a defect of a bone of a head of a subject;
(b) a recess in the porous body, wherein the recess comprises a dimension larger than an average pore dimension of the porous body; and
(c) a biologic disposed in the recess.

8. The implant of claim 7, wherein the biologic comprises demineralized bone matrix, morselized bone, bone marrow aspirate, bone morphogenetic protein 2 (BMP2), beta-TCP, or any combination thereof.

9. The implant of claim 7, wherein the recess comprises a curved surface.

10. The implant of claim 7, wherein the recess forms a channel from one end of the porous body to another end of the porous body.

11. The implant of claim 7, wherein the recess comprises an opening that opens inwards to the subject.

12. The implant of claim 7, wherein the recess comprises a plurality of recesses.

13. The implant of claim 12, wherein each of the plurality of recesses comprises different dimensions.

14. The implant of claim 7, wherein the porous body comprises a porosity of at least 25, 30, 35, 40, 45, 50, 55, 60, or 65%.

15. An implant, comprising:
(a) a porous body comprising a form that fills a defect of a bone of a head of a subject;
(b) a biologic disposed in the porous body; and
(c) an extension connected to the porous body, wherein the extension is configured to lay against the bone of the head of the subject.

16. The implant of claim 15, wherein the extension comprises an attachment opening.

17. The implant of claim 16, wherein the extension comprises a plurality of attachment openings.

18. The implant of claim 17, wherein the plurality of attachment openings are oriented at an angle with respect to another.

19. The implant of claim 15, wherein the implant comprises a plurality of extensions.

20. The implant of claim 15, wherein the porous body comprises a porosity of at least 25, 30, 35, 40, 45, 50, 55, 60, or 65%.
